(19) United States
(12) Patent Application Publication
(10) Pub. No.: US 2025/0290023 A1
(43) Pub. Date: Sep. 18, 2025

(54) High-Throughput Mechanoporation for Scalable and Cost-Effective Cell Therapy Manufacturing

(71) Applicant: CellFE, Inc., Alameda, CA (US)

(72) Inventors: Sewoon Han, Albany, CA (US); Alexander Alexeev, Atlanta, GA (US); Ockchul Kim, Alameda, CA (US)

(21) Appl. No.: 19/079,840

(22) Filed: Mar. 14, 2025

Related U.S. Application Data
(60) Provisional application No. 63/565,910, filed on Mar. 15, 2024.
(60) Provisional application No. 63/654,433, filed on May 31, 2024.

(51) Int. Cl. C12M 1/42; C12M 1/00; C12M 1/34

(52) U.S. Cl. CPC C12M 35/04; C12M 23/02; C12M 23/34; C12M 23/38; C12M 29/14; C12M 29/20; C12M 29/26; C12M 41/40

(57) ABSTRACT

Described herein are high-throughput, high-volume mechanoporation systems and methods for intracellular payload delivery, enabling scalable and cost-effective cell therapy manufacturing. The apparatus includes a pre-processing media reservoir with a media cavity and a processing stack comprising two end gaskets and a processing unit. The processing unit features inlet and outlet slots, processing channels fluidically connecting them, and ridges forming gaps within the channels. The inlet slots align with gasket slots in the end gasket, allowing media to flow toward the ridges. When pressure is applied to the media reservoir, the media is forced through the gaps in the processing channels, inducing mechanoporation of cells. This system automates and streamlines cell engineering, improving throughput, consistency, and scalability while reducing costs, aligning with the needs of advanced cell therapy production.

### CROSS-REFERENCE TO RELATED APPLICATIONS

This application claims the benefit under 35 U.S.C. § 119(e) of U.S. Provisional Patent Application 63/565,910, filed on 2024 Mar. 15 (Ref: CLFEP012P) and U.S. Provisional Patent Application 63/654,433, filed 2024 May 31 (Ref: CLFEP012P2), both of which are incorporated herein by reference in their entirety for all purposes.

### BACKGROUND

Intracellular delivery has many valuable applications, such as gene transfection, editing, cell labeling, and cell interrogation. However, conventional delivery methods (e.g., microinjection, electroporation, chemical poration, and sonoporation) have demonstrated low delivery efficiencies and cell viability, especially for large molecules (e.g., molecules with sizes of at least 2000 kDa) and large particles (e.g., particles with sizes of at least 50 nanometers). Furthermore, many conventional delivery methods are not able to process cells at high rates (e.g., 10\hat{ }8 cells per second), high volumes (e.g., 1-100 milliliters of media solution per batch), and large amounts of biological cells (e.g., 10\hat{ }9 cells per batch). For example, cells often require individual handling, which significantly slows down processing speeds. What is needed are new methods and systems for high-throughput payload delivery into biological cells.

### SUMMARY

Described herein are high-throughput, high-volume mechanoporation systems and methods for intracellular payload delivery, enabling scalable and cost-effective cell therapy manufacturing. The apparatus includes a pre-processing media reservoir with a media cavity and a processing stack comprising two end gaskets and a processing unit. The processing unit features inlet and outlet slots, processing channels fluidically connecting them, and ridges forming gaps within the channels. The inlet slots align with gasket slots in the end gasket, allowing media to flow toward the ridges. When pressure is applied to the media reservoir, the media is forced through the gaps in the processing channels, inducing the mechanoporation of cells. This system automates and streamlines cell engineering, improving throughput, consistency, and scalability while reducing costs, and aligning with the needs of advanced cell therapy production.

Clause 1. A high-throughput high-volume mechanoporation unit for scalable and cost-effective cell therapy manufacturing, the high-throughput high-volume mechanoporation unit comprising: a first enclosure portion comprising a media inlet; a pre-processing media reservoir comprising a media cavity forming a delivery slot, wherein: the media cavity is fluidically coupled with the media inlet, and the pre-processing media reservoir is sealed against the first enclosure portion; a processing stack comprising two end gaskets and a processing unit positioned between the two end gaskets, wherein: each of the two end gaskets comprises a set of gasket slots, fluidically coupled to the delivery slot, the processing unit comprises a set of inlet slots, a set of outlet slots, a set of processing channels extending between and fluidically coupling the set of inlet slots and the set of outlet slots, and a set of ridges extending into the set of processing channels and forming a set of gaps within the set of processing channels, the set of inlet slots is aligned with the set of gasket slots in a first one of the two end gaskets, and the second processing slot is aligned with one slot in the set of gasket slots in the second one of the two end gaskets; a stack support interfaces the second one of the two end gaskets providing support to the processing stack when a pressure differential is applied across the processing stack; and a second enclosure portion comprising a cavity fluidically coupled to the set of outlet slots and used for collecting the cell media after passing through the processing stack.

Clause 2. The high-throughput high-volume mechanoporation unit of clause 1, wherein each the set of gasket slots extends perpendicular to the delivery slot.

Clause 3. The high-throughput high-volume mechanoporation unit of clause 1, wherein: the first one of the two end gaskets is positioned between and sealed against each of the processing unit and the pre-processing media reservoir, and the second one of the two end gaskets is positioned between and sealed against each of the processing unit and the.

Clause 4. The high-throughput high-volume mechanoporation unit of clause 1, wherein: the set of inlet slots does not overlap with the set of gasket slots in a second one of the two end gaskets, and the second processing slot does not overlap with any slot in the set of gasket slots in the first one of the two end gaskets and is sealed by the first one of the two end gaskets.

Clause 5. The high-throughput high-volume mechanoporation unit of clause 1, wherein: the stack support comprises a set of rib separated by a set of openings, the set of rib provides support to the processing stack, and the set of openings is aligned with the set of gasket slots in the second one of the two end gaskets.

Clause 6. The high-throughput high-volume mechanoporation unit of clause 1, wherein: the second enclosure portion comprises a media outlet and a gas outlet, both fluidically coupled to the cavity, and the gas outlet is located closer to the processing stack than the media outlet.

Clause 7. The high-throughput high-volume mechanoporation unit of clause 1, wherein the first enclosure portion comprises a first enclosure cavity such that the pre-processing media reservoir protrudes into the first enclosure cavity and seals to a bottom of the first enclosure cavity.

Clause 8. The high-throughput high-volume mechanoporation unit of clause 1, wherein: the media cavity comprises a rectangular portion and a taper portion, the rectangular portion faced and is fluidically coupled with the media inlet, and the taper portion terminates with the delivery slot.

Clause 9. The high-throughput high-volume mechanoporation unit of clause 1, wherein the media cavity has a volume of 10 milliliters to 50 milliliters.

Clause 10. The high-throughput high-volume mechanoporation unit of clause 1, wherein: the processing unit comprises a silicon base and a glass cover attached to the silicon base and forming the set of processing channels with the silicon base, and the set of ridges is a part of the silicon base.

Clause 11. The high-throughput high-volume mechanoporation unit of clause 10, wherein the glass cover is attached to the silicon base using anodic bonding.

Clause 12. The high-throughput high-volume mechanoporation unit of clause 10, wherein: the processing stack comprises a second processing unit comprising a second silicon base and a second glass cover attached to the second silicon base, and the processing stack comprises an intermediate gasket providing a seal between the glass cover and the second silicon base.

Clause 13. The high-throughput high-volume mechanoporation unit of clause 12, wherein the intermediate gasket is formed by casting silicone rubber and compressed between the glass cover and the second silicon base.

Clause 14. The high-throughput high-volume mechanoporation unit of clause 12, wherein: the glass cover comprises a planar surface directly interfacing the second silicon base, and the glass cover further comprises a seal channel recessed from the planar surface and housing the intermediate gasket.

Clause 15. The high-throughput high-volume mechanoporation unit of clause 14, wherein: the seal channel extends to an edge of the glass cover forming a set of seal channel openings, and the intermediate gasket is formed in-situ by flowing a gasket material through the set of seal channel openings to fill the seal channel with the gasket material, while the planar surface of the glass cover directly interfaces the second silicon base, and curing the gasket material inside the seal channel.

Clause 16. The high-throughput high-volume mechanoporation unit of clause 15, wherein the gasket material is selected from the group consisting of a heat-curable material and a ultraviolet (UV) curable material.

Clause 17. The high-throughput high-volume mechanoporation unit of clause 10, wherein: the processing unit comprises a processing-unit first surface formed by the glass cover and directly interfacing the first one of the two end gaskets, the processing unit comprises a processing-unit second surface formed by the glass cover, and each of the set of inlet slots and the set of outlet slots extends between the processing-unit first surface and the processing-unit second surface.

Clause 18. The high-throughput high-volume mechanoporation unit of clause 10, wherein: one slot of the set of inlet slots extends between a first one of the set of outlet slots and a second one of the set of outlet slots, a first subset of the set of processing channels extends between the one slot of the set of inlet slots and the first one of the set of outlet slots, and a second subset of the set of processing channels extends between the one slot of the set of inlet slots and the second one of the set of outlet slots.

Clause 19. The high-throughput high-volume mechanoporation unit of clause 1, wherein each of the set of processing channels has 1 to 5 ridges of the set of ridges.

Clause 20. The high-throughput high-volume mechanoporation unit of clause 1, wherein each ridge of the set of ridges has a chevron shape with a leading chevron edge facing a corresponding one of the set of inlet slots.

Clause 21. The high-throughput high-volume mechanoporation unit of clause 1, wherein each of the set of processing channels has a length, defined by a distance between a corresponding one of the set of inlet slots and a corresponding one of the set of outlet slots, of 1 millimeter to 10 millimeters.

Clause 22. The high-throughput high-volume mechanoporation unit of clause 1, wherein: the stack support comprises a stack-facing surface that interfaces the second one of the two end gaskets, and a set of openings taper away from the stack-facing surface.

Clause 23. The high-throughput high-volume mechanoporation unit of clause 1, wherein the second enclosure portion is attached to the first enclosure portion.

Clause 24. A method of processing cells in a cell media using a high-throughput high-volume mechanoporation unit comprising a first enclosure portion, a pre-processing media reservoir, a processing stack, a stack support, and a second enclosure portion, the method comprising: delivering the cell media to the pre-processing media reservoir; increasing air pressure in the pre-processing media reservoir over the media to at least 5 psi (34 kPa) while monitoring an airflow rate through a gas outlet of the second enclosure portion; reducing the air pressure in the pre-processing media reservoir to atmospheric level as soon the airflow rate through the gas outlet reaches at least a set limit; and removing the media through a media outlet of the second enclosure portion.

Clause 25. The method of clause 24, wherein delivering media to the pre-processing media reservoir comprises flowing the media, under static pressure from the cell media, to a set of ridges.

Clause 26. The method of clause 24, wherein the media has a cell density of at least 20×10\hat{ }6 cells per milliliter.

### BRIEF DESCRIPTION OF THE DRAWINGS

The included drawings are for illustrative purposes and serve only to provide examples of possible structures and operations for the disclosed inventive systems, apparatus, and methods. These drawings in no way limit any changes in form and detail that may be made by one skilled in the art without departing from the spirit and scope of the disclosed implementations.

FIG. 1A is an exploded view of a high-throughput high-volume mechanoporation unit illustrating various components and their arrangement in the apparatus, in accordance with some examples.

FIG. 1B is an exploded view of another example of a high-throughput high-volume mechanoporation unit.

FIGS. 2A and 2B are different perspective views of the first enclosure portion of the high-throughput high-volume mechanoporation unit in FIG. 1A, in accordance with some examples.

FIGS. 3A, 3B, 3C, and 3D are different perspective, top, and cross-sectional views of the pre-processing media reservoir of the high-throughput high-volume mechanoporation unit in FIG. 1A, in accordance with some examples.

FIG. 4A is a side cross-sectional view of a processing stack formed by two end gaskets and a processing unit positioned between the two end gaskets, in accordance with some examples.

FIG. 4B is a side cross-sectional view of another processing stack formed by two end gaskets, two processing units, and one intermediate gasket such that each processing unit is positioned between the intermediate gasket and one of the end gaskets, in accordance with some examples.

FIGS. 4C and 4D are the top views of the end gasket and the intermediate gasket in FIG. 4E, in accordance with some examples.

FIG. 4E is a perspective view of an end gasket of the high-throughput high-volume mechanoporation unit in FIG. 1A, in accordance with some examples.

FIGS. 4F is a top view of the end gasket in FIG. 4A, in accordance with some examples.

FIGS. 4G is a perspective view of another example of the processing stack.

FIG. 4H is a bottom perspective view of the end gasket in the processing stack of FIG. 4G.

FIG. 4I is a cross-sectional schematic side view of a processing stack with two processing units, each comprising a silicon base (forming a ridge) and a glass cover attached to the silicon base and comprising a seal channel housing an intermediate gasket, in accordance with some examples.

FIGS. 4J and 4K are side and top views of the processing stack in FIG. 4I.

FIG. 4L is a schematic view of two processing units prior to forming a stack and filling a seal channel with an intermediate gasket, in accordance with some examples.

FIG. 4M is a cross-sectional view of a processing unit illustrating various features of the silicon base and the glass cover.

FIG. 4N is a perspective view of a processing unit illustrating various features of the silicon base and the glass cover.

FIG. 5A is a top cross-sectional view of a processing unit for use in a high-throughput high-volume mechanoporation unit, in accordance with some examples.

FIG. 5B is a top cross-sectional view of a portion of the processing unit in FIG. 5A, in accordance with some examples.

FIG. 5C is a top cross-sectional view of a processing channel in the processing unit in FIG. 5A, in accordance with some examples.

FIG. 5D is a front view of a processing channel in the processing unit in FIG. 5A, in accordance with some examples.

FIGS. 5E is a cross-sectional view of the processing channel, in accordance with some examples.

FIGS. 5F is a top view of the processing channel, in accordance with some examples.

FIGS. 6A, 6B, 6C, and 6D are different perspective, top, and cross-sectional views of the stack support of the high-throughput high-volume mechanoporation unit in FIG. 1A, in accordance with some examples.

FIGS. 7A and 7B are different perspective views of the second enclosure portion of the high-throughput high-volume mechanoporation unit in FIG. 1A, in accordance with some examples.

FIG. 8A is a block diagram of a system comprising a high-throughput high-volume mechanoporation unit, in accordance with some examples.

FIGS. 8B-8M are schematic cross-sectional views of different examples of the high-throughput high-volume mechanoporation unit comprising a media isolator. FIG. 9 is a process flowchart corresponding to a method of operating a high-throughput high-volume mechanoporation unit, in accordance with some examples.

FIG. 10 is a plot of the inlet pressure as a function of time during the operation of a high-throughput high-volume mechanoporation unit, in accordance with some examples.

FIGS. 11A-11G are schematic views of another example of a high-throughput high-volume mechanoporation unit.

### DETAILED DESCRIPTION

### Introduction

As noted above, cell processing methods and systems are based on a mechanoporation approach, in which a population of cells is rapidly compressed and then released to relax and absorb the payload from the surrounding media. Unlike membrane shearing and other conventional methods, the compression used in mechanoporation generally occurs in less than 1 second, often within 10 microseconds to 300 milliseconds. The compression time depends on the flow rate, cell size, ridge geometry, and other factors. Without being restricted to any particular theory, it is believed that such a rapid compression causes the cells to transiently change their volume through membrane pores (i.e., experience temporal volume loss of at least 20% or even at least 30%) rather than simply changing their shapes to adapt to the ridge gaps. The ridge gaps are sometimes referred to as constrictions. The compressed state is a non-natural state for the cells and the cells will attempt to recover to the original volume.

After the compression and volume reduction, the cells are released into a recovery space where the cells are no longer compressed and are allowed to recover. Specifically, the volume of the cells can increase back to normal by absorbing the surrounding media. The media, which surrounds the cells, comprises various payloads (e.g., reagents). These payloads are introduced into the cells as a part of this recovery. For example, plasmids and magnetic nanoparticles have been successfully introduced into stem cells using this technique, and plasmids and mRNA have been introduced into primary peripheral blood mononuclear cells. It should be noted that this technique is suitable for large reagents that generally cannot be introduced by other microfluidic methods, such as squeezing cells in narrow pores that can lead to mechanical membrane poration followed by diffusion of reagents suspended in the media through the membrane pores. Since the diffusion is slower for larger reagents, such cannot be effectively delivered by diffusion into cells due to natural membrane pore closure. Furthermore, the degree of cell membrane poration proposed by conventional methods is limited to ensure cell viability and avoid cell damage or death. Finally, conventional microfluidic devices for intracellular delivery are prone to clogging because narrow channels are used for achieving high membrane shear and creating membrane pores. However, increasing flow rates to reduce clogging usually results in cell damage and cell death.

The process of compression and recovery may be performed multiple times, e.g., once for each ridge inside the microfluidic channel and space between ridges or ridge and outlet. More specifically, each ridge forms a ridge gap with an opposing wall of the apparatus and the cells are compressed while passing through each one of these ridge gaps. In some examples, the ridges may form different gaps. Furthermore, the ridge gaps can be adjusted, e.g., by moving different components of the cell processing apparatus relative to each other. This gap adjustment may be used, e.g., to reconfigure the apparatus (e.g., to process different types of cells), prevent clogging, cleaning, and other like purposes. It should be noted that changing the ridge spacing affects the degree, speed, and/or duration of cell compressions. For example, decreasing the gap formed by sequential ridges allows for achieving higher levels of compression. Cells recover when passing in the recovery space between consecutive ridges or after the last ridge and the channel outlet. During the recovery cells absorb surrounding media with payload.

In general, the methods and systems described herein may be used to deliver a variety of macromolecules to a variety of different cell types. The intracellular delivery is achieved with high throughput and minimal clogging while posing a lower risk of cell death and aggregation than conventional methods.

The benefit of high-throughput high-volume mechanoporation units is the parallel processing of a significantly increased number of cells using the same uniform conditions. The throughput is determined by the number of processing channels that are fluidically coupled parallel to each other and share effectively the same inlet (formed by fluidically coupled inlets) and the same outlet (formed by fluidically coupled outlets). The fluidic coupling of inlets and outlets provides substantially less flow resistance than the processing channels, e.g., due to their relative size and presence of ridges in the processing channels. This feature ensures the uniformity of processing conditions in all processing channels, e.g., the pressure differential across these channels.

One inherent issue with mechanoporation-type cell processing devices is clogging. Cell media typically includes a number of non-compressible cells, which are stuck with the gap formed by a ridge effectively blocking the channel. The number of cells that can pass through a single channel, before the channel is blocked, may be referred to as a channel cell-processing capacity. The channel cell-processing capacity depends on the channel design (e.g., the gap size, width, number of ridges, shape of ridges, etc.) and cell media properties (e.g., the cell size, cell density, cell viability, etc.). In various examples, the channel cell-processing capacity is 500,000-5,000,000 cells (for each channel). Using processing channels with a width that is significantly larger than channel height (e.g., at least 5 times larger, at least 20 times larger) allows for processing of the cell media with high cell density. While some cells flown in the channel slow the velocity as the cells are compressed by a ridge, other cells can pass around the slow cells preventing cells from piling up and clogging the channel. For example, the passing cells can be more compressible and/or can follow travel to other parts of the same ridge (e.g., in a chevron ridge orientation). In various examples, the channels can process cell samples with cell density of greater than 5 million cells per milliliter or even greater than 50 million cells per milliliter such as between 1-200 million cells per milliliter or, more specifically, between 30-150 million cells per milliliter.

Using multiple processing channels in parallel allows for addressing this channel-clogging issue. At the same time, a high-throughput high-volume mechanoporation unit is designed in such a way that the media supplied into the apparatus is not wasted, i.e., enough processing channels remain operational until all batched-supplied media is processed. Using multiple processing channels in parallel allows the processing of samples with high volume. The volume of the sample depends on the number of channels, channel processing capacity, and cell density. In various examples, the volume of a cell media sample can be at least 50 milliliters, at least 200 milliliters, or even at least 1 liter. For purposes of this description, the term “high-throughput high-volume” (in the context of a mechanoporation apparatus) is defined as a mechanoporation apparatus capable of processing the cell media sample volume specified above at the cell concentrations also specified above.

### High-Throughput High-Volume Mechanoporation Unit Examples

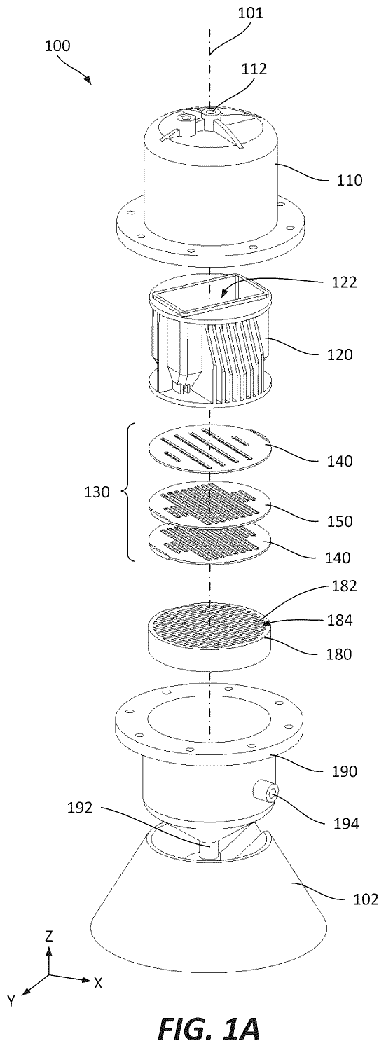

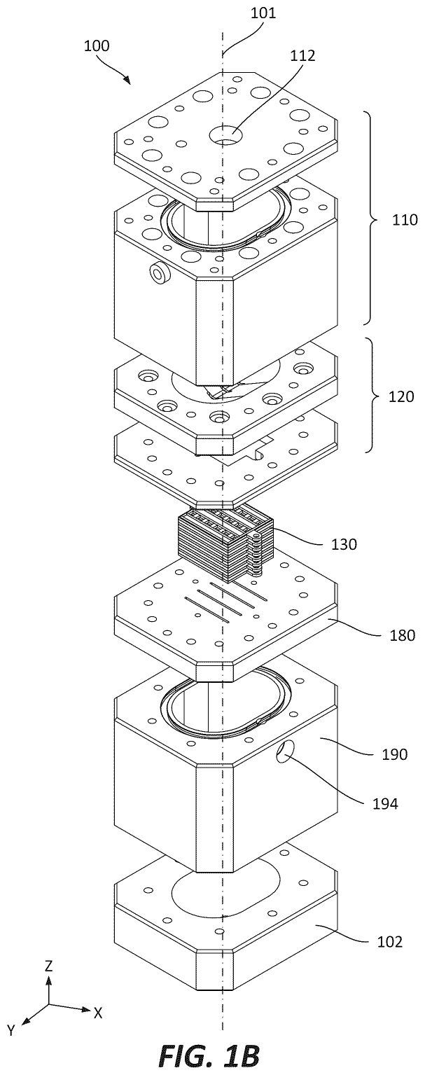

FIG. 1A is an exploded view of a high-throughput high-volume mechanoporation unit 100 illustrating various components and their arrangement in the apparatus, in accordance with some examples. Specifically, in the illustrated example, the high-throughput high-volume mechanoporation unit 100 comprises a first enclosure portion 110, a pre-processing media reservoir 120, a processing stack 130 comprising two end gaskets 140, and a processing unit 150, a stack support 180, and a second enclosure portion 190. FIG. 1A also illustrates a stand 102, which is configured to engage the second enclosure portion 190 to keep the high-throughput high-volume mechanoporation unit 100 in the upright position, i.e., such that the primary axis 101 of the high-throughput high-volume mechanoporation unit 100 is aligned with/parallel to the gravitation vertical. FIG. 1B is an exploded view of another example of a high-throughput high-volume mechanoporation unit 100.

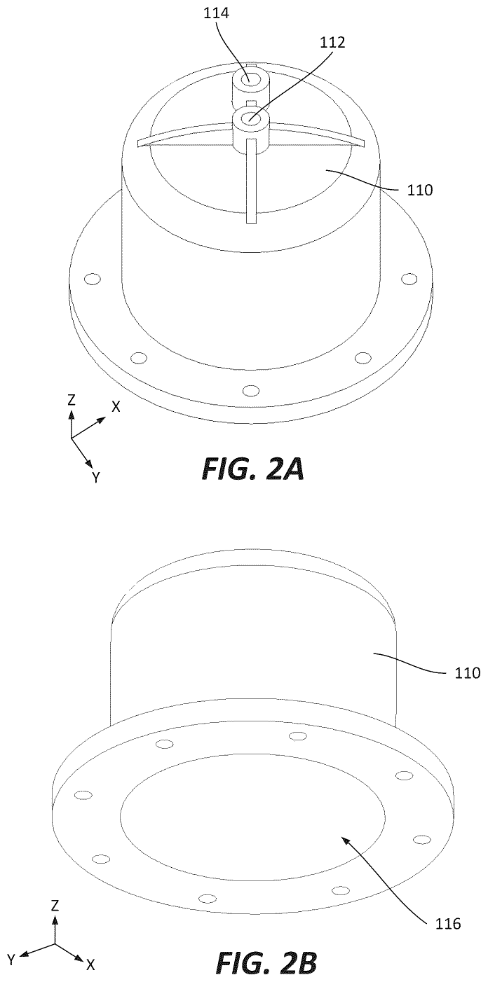

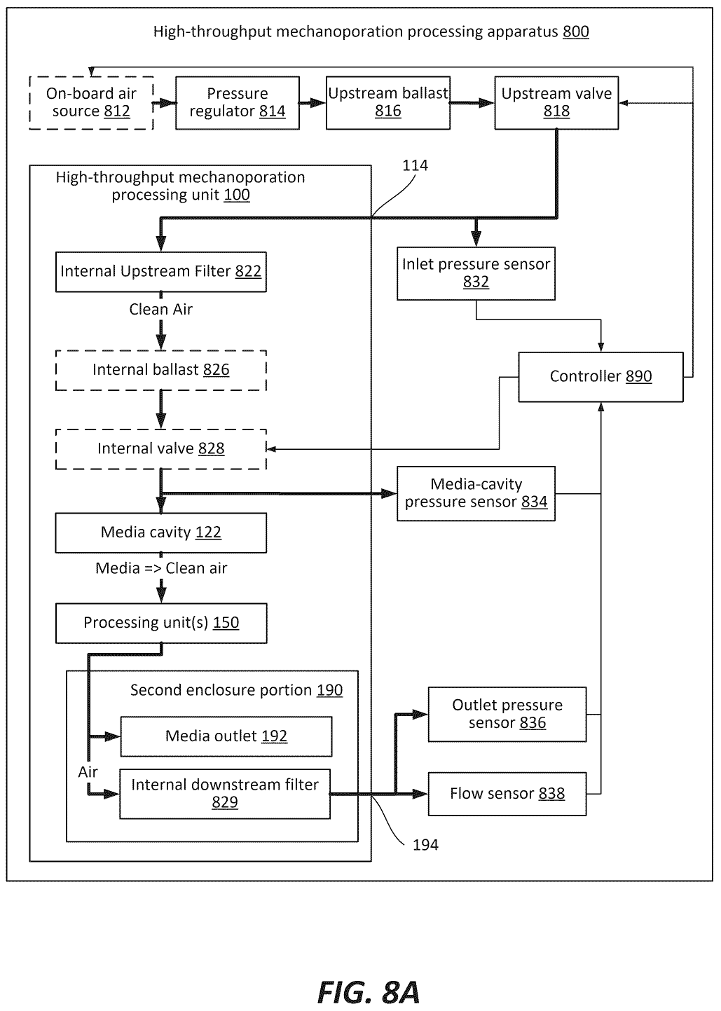

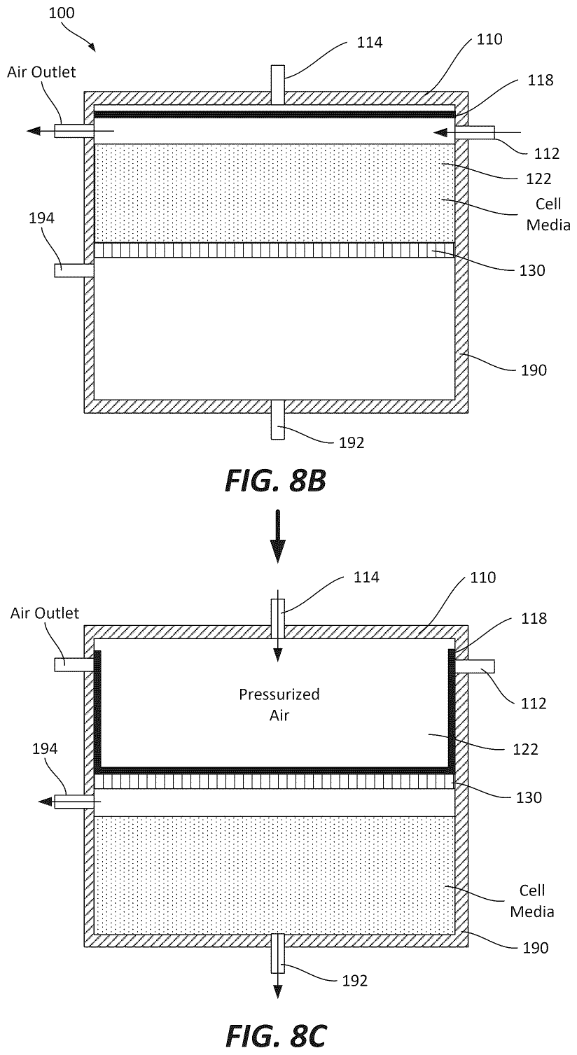

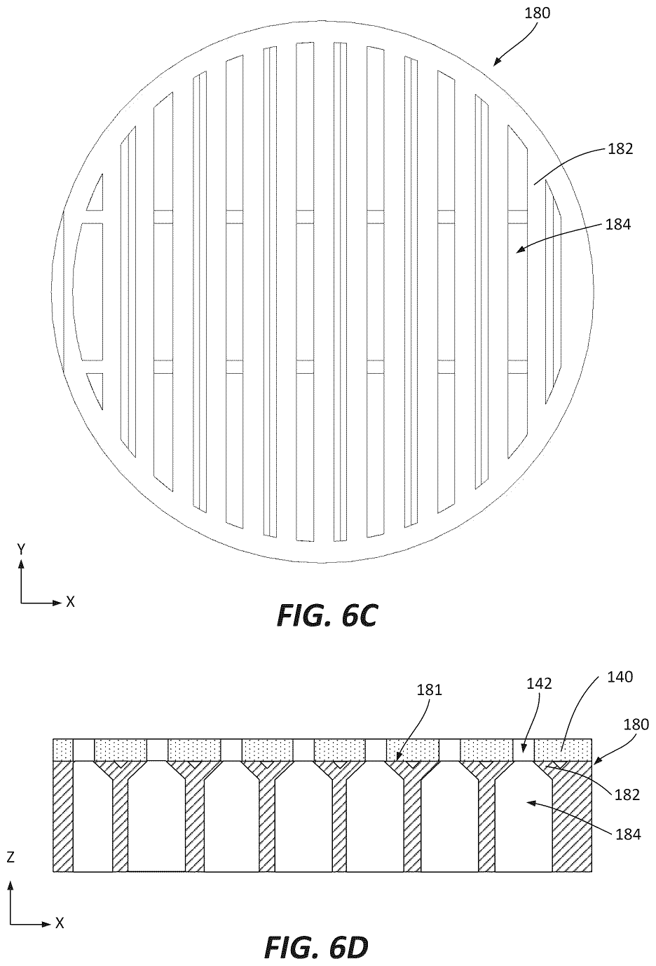

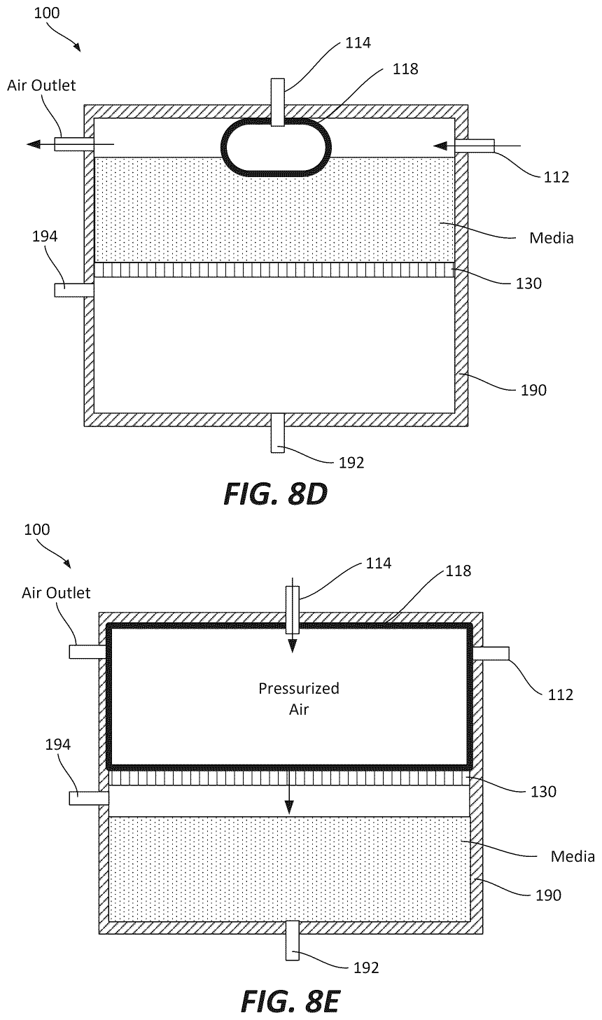

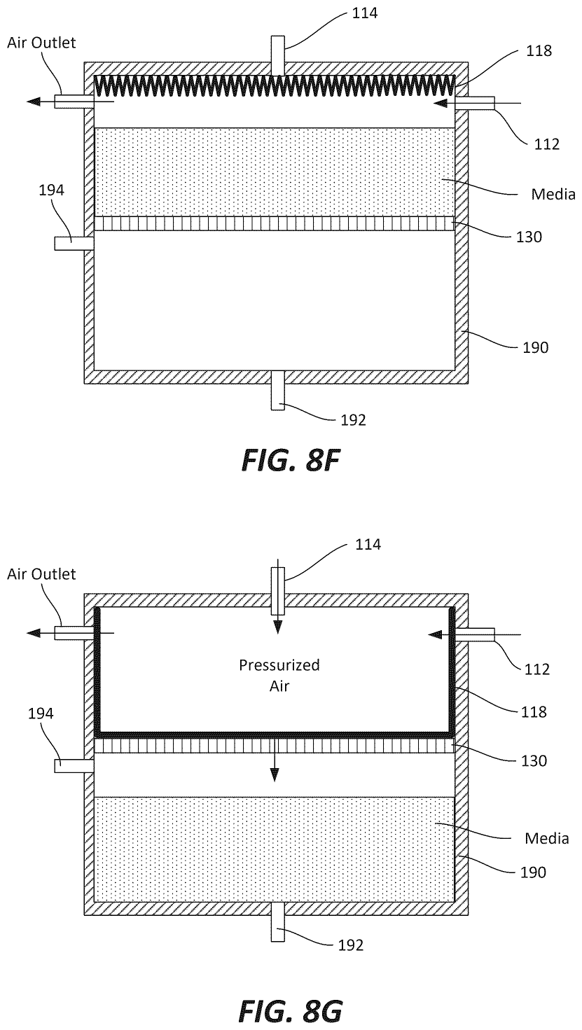

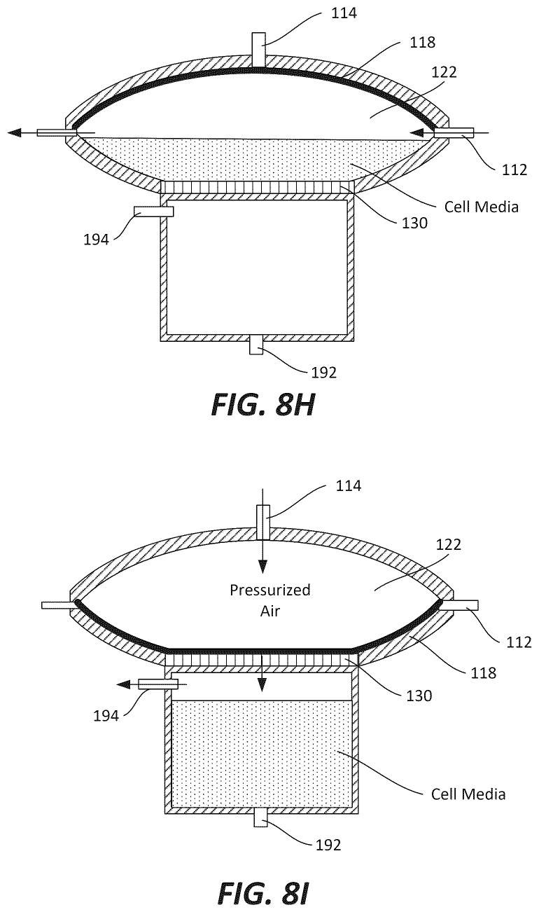

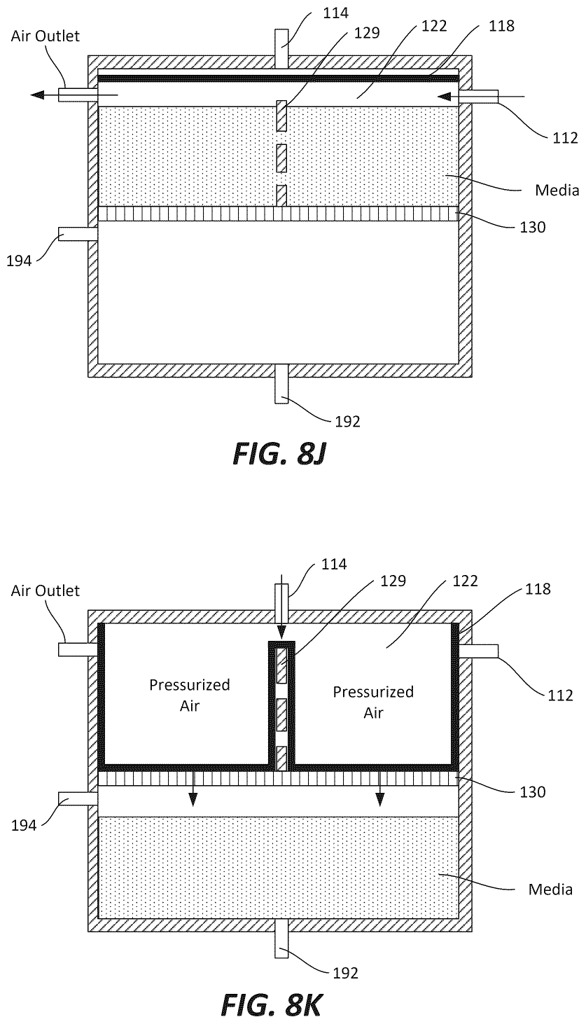

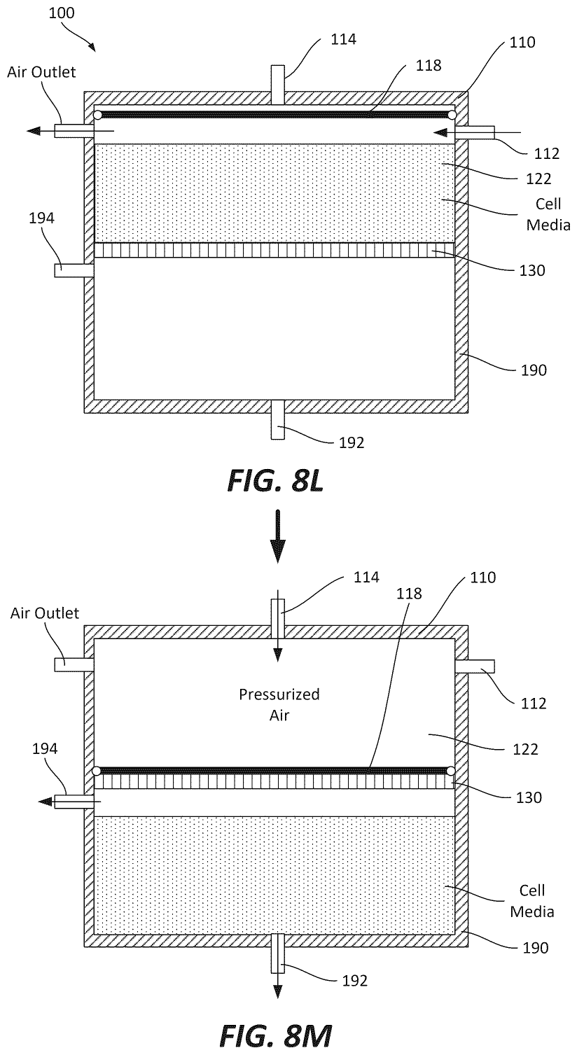

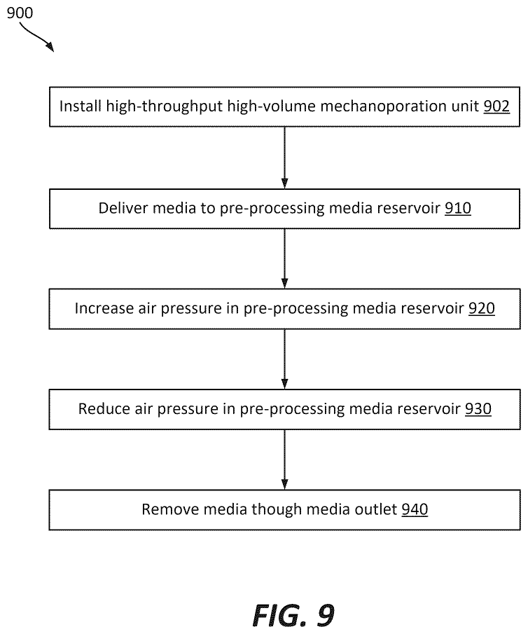

Referring to FIGS. 2A and 2B, the first enclosure portion 110 comprises a media inlet 112 for delivering media into the high-throughput high-volume mechanoporation unit 100. Various examples of the media and delivery operation are described below with reference to FIG. 8A. The media inlet 112 may be equipped with a valve to isolate the interior of the high-throughput high-volume mechanoporation unit 100 from the environment and/or to connect the interior to a pressure source. In some examples, the first enclosure portion 110 also comprises a gas inlet 114, which also provides fluidic coupling to the interior. In these examples, the media inlet 112 is used to deliver the media, after which the media inlet 112 is sealed. During the media delivery, gas inlet 114 may be used for venting (i.e., allowing the air, displaced by the media, to escape from the interior) thereby preventing bubbling/foaming of the media as the media is being delivered through the media inlet 112. After the media delivery and sealing of the media inlet 112, the gas inlet 114 is then used to pressurize the media or, more specifically, a portion of the interior of the high-throughput high-volume mechanoporation unit 100 positioned above the media surface.

The first enclosure portion 110 may also comprise a first enclosure cavity 116 such that the pre-processing media reservoir 120 protrudes into the first enclosure cavity 116 and seals to the bottom of the first enclosure cavity 116. As noted above, the cavity bottom also supports the media inlet 112. The first enclosure cavity 116 is formed by sidewalls (e.g., which may have a cylindrical shape) that terminate with flanges for attachment to the second enclosure portion 190. It should be noted that the attachment between the first enclosure cavity 116 and the second enclosure portion 190 is used to maintain various internal seals while the pressure differential across the seals can reach/exceed 100 psi (689 kPA).

The first enclosure portion 110 and the second enclosure portion 190 can be formed from various biocompatible materials such as polycarbonate, polystyrene, cyclic, and olefin copolymer (COC). These materials provide sufficient structural integrity to the high-throughput high-volume mechanoporation unit 100.

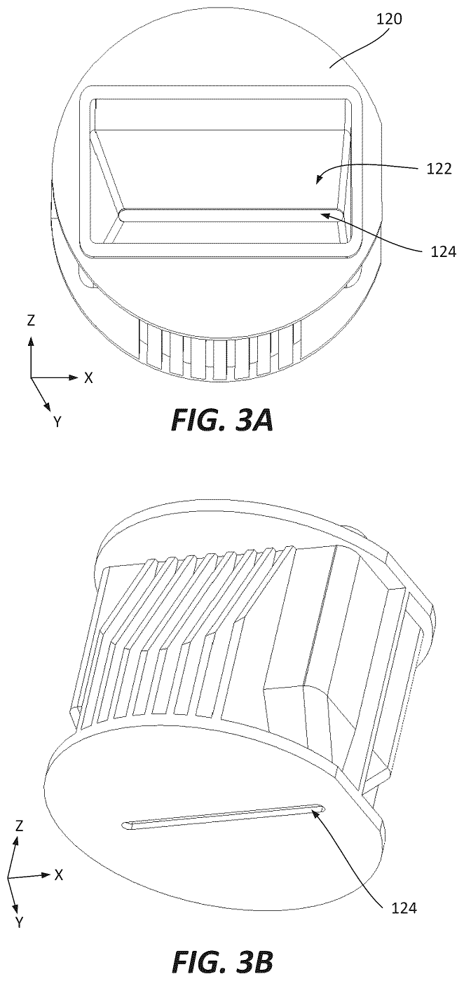

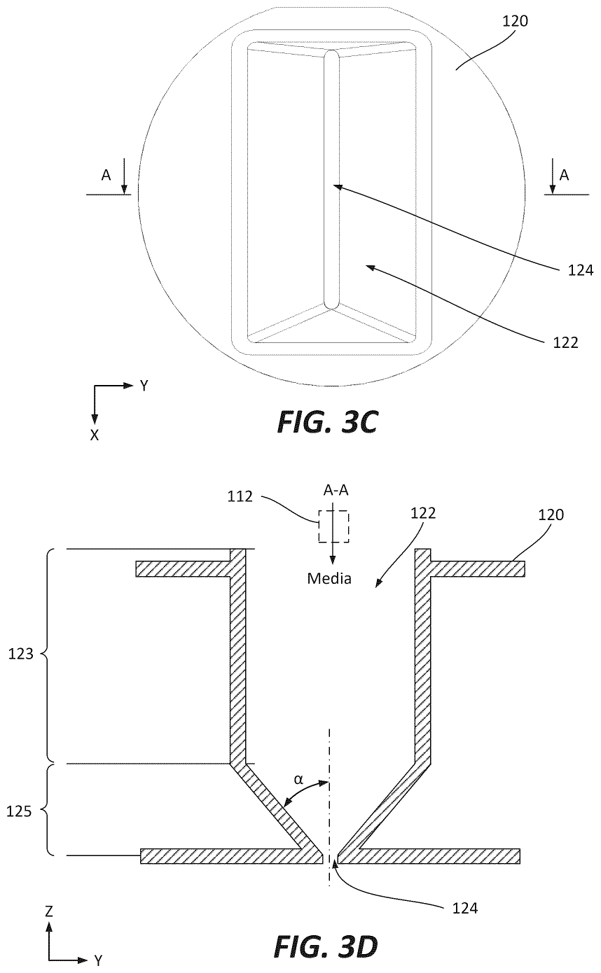

Referring to FIGS. 3A and 3B, the pre-processing media reservoir 120 comprises a media cavity 122 forming a delivery slot 124. The media cavity 122 is fluidically coupled with the media inlet 112. In other words, when media is delivered through the media inlet 112, the media is collected in the media cavity 122. The pre-processing media reservoir 120 is sealed against the first enclosure portion 110 (e.g., the bottom of the first enclosure cavity 116) thereby allowing to pressurize the interior portion about the media surface and push the media through the delivery slot 124.

Referring to FIGS. 3C and 3D, the media cavity 122 comprises a rectangular portion 123 and a taper portion 125. The rectangular portion 123 faces and is fluidically coupled with the media inlet 112. The taper portion 125 terminates with the delivery slot 124 and is positioned between the rectangular portion 123 and the delivery slot 124. The taper portion 125 allows maintaining the delivery slot 124 covered by the media (e.g., maintain a certain minimum depth of the media) as the volume of the media inside the media cavity 122 decreases (displaced through the delivery slot 124 during the operation of the high-throughput high-volume mechanoporation unit 100). In other words, the taper portion 125 reduces the “dead volume” in the media cavity 122, which corresponds to the media volume that remains in the media cavity 122 after the operation stops. In some examples, the dead volume represents less than 10% of the total volume of the media cavity 122. It should be noted that in addition to the design of the media cavity 122, the dead volume is influenced by surface tension, pressure, surface chemistry, and other operating factors.

This ensures that the air pushing on the media does not reach the delivery slot 124 up until most of the media (e.g., at least 90% or even at least 95%) is displaced from the media cavity 122. Once the air gets into the delivery slot 124, the air can find the least resistive path through the high-throughput high-volume mechanoporation unit 100 from the gas inlet 114 to the gas outlet 194, which effectively ends the operation of the high-throughput high-volume mechanoporation unit 100.

Referring to FIG. 3D, in some examples, the angle (a) of the sidewalls in the taper portion 125 relative to the primary axis 101 is 15°-60° or, more specifically, 20°-40°. The larger angle helps to maximize the volume of the media cavity 122, while the smaller angle helps to maintain the delivery slot 124 covered by the media. In some examples, the height ratio (along the Z direction) of the rectangular portion 123 to the taper portion 125 is 1.5-4 or, more specifically, 2-3.

In some examples, the media cavity 122 has a volume of 1 milliliter to 100 milliliters or, more specifically, 10 milliliters to 50 milliliters. The volume depends on the volume of media and the total number of processing channels in the processing stack 130 (which can include one or more processing units 150) and the processing capacity of each channel (defined by various characteristics of the channel and the media further described below). The volume of the media cavity needs to match the volume of the media to reduce transient time due to the pressurization of the gas in the media cavity.

### Examples of Intermediate and End Gaskets

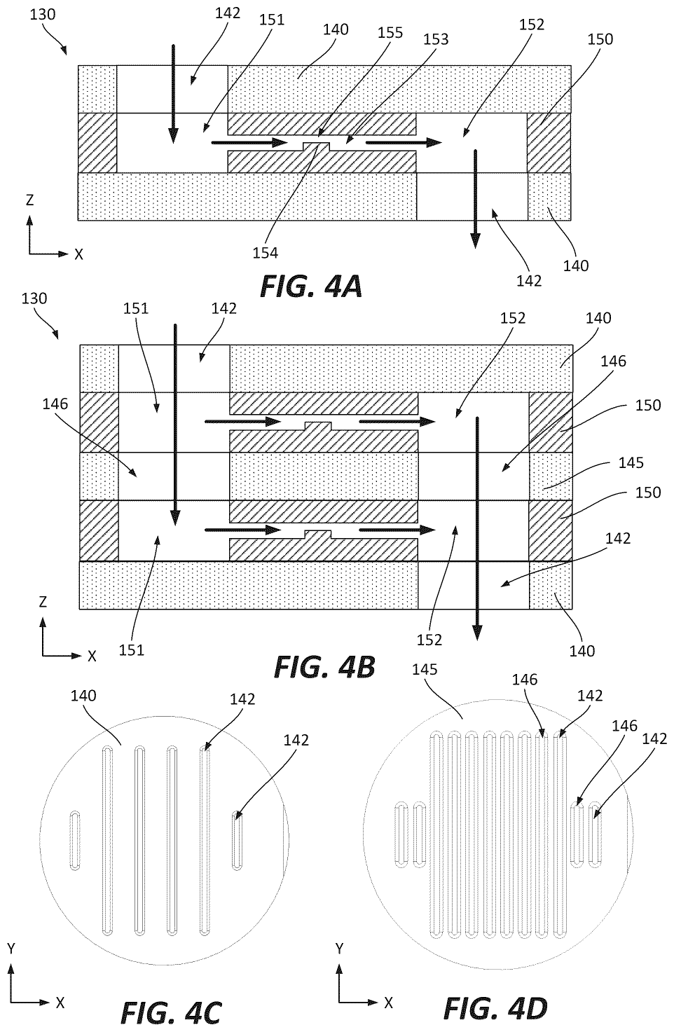

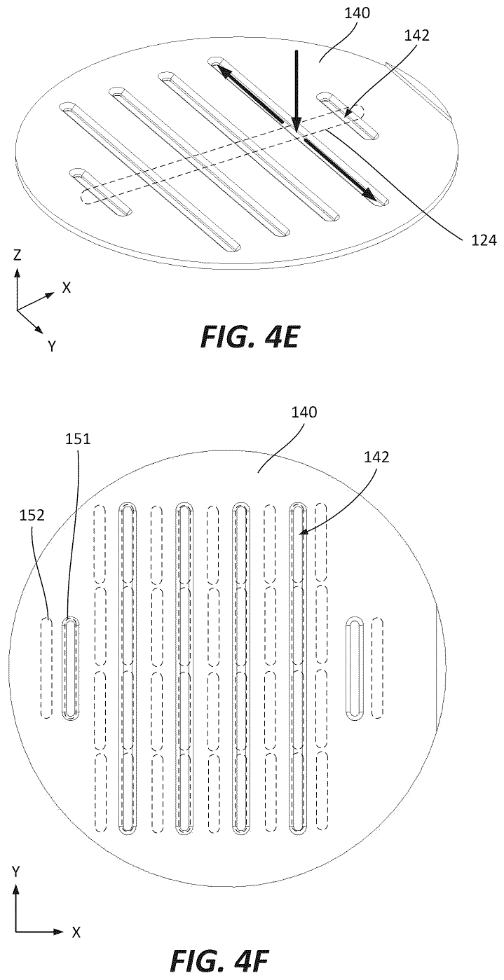

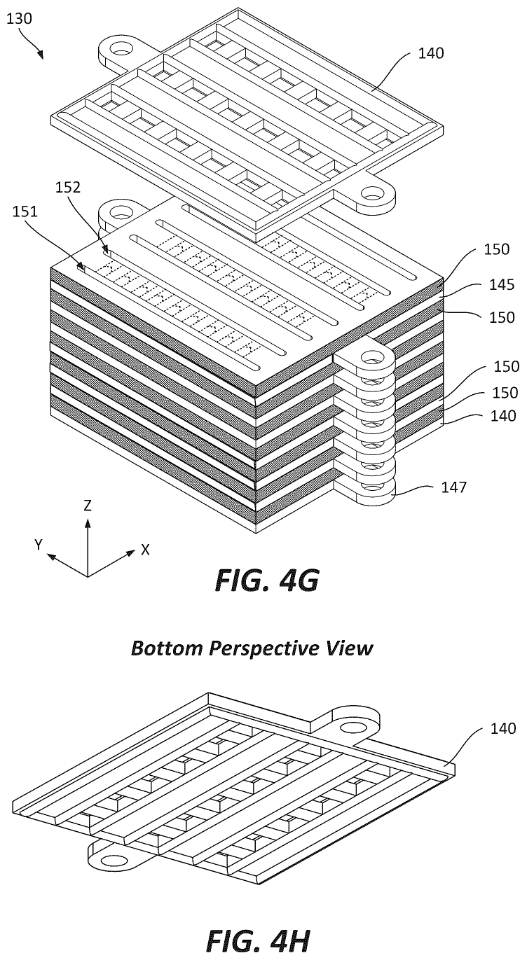

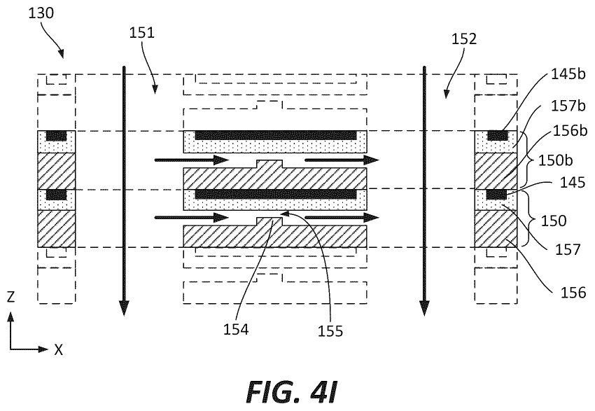

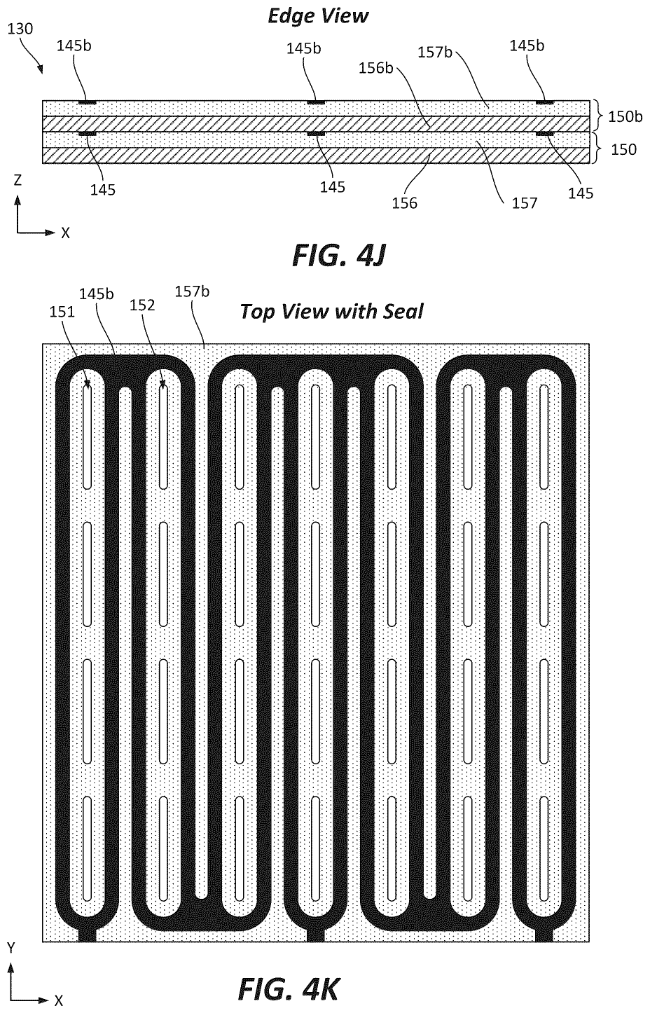

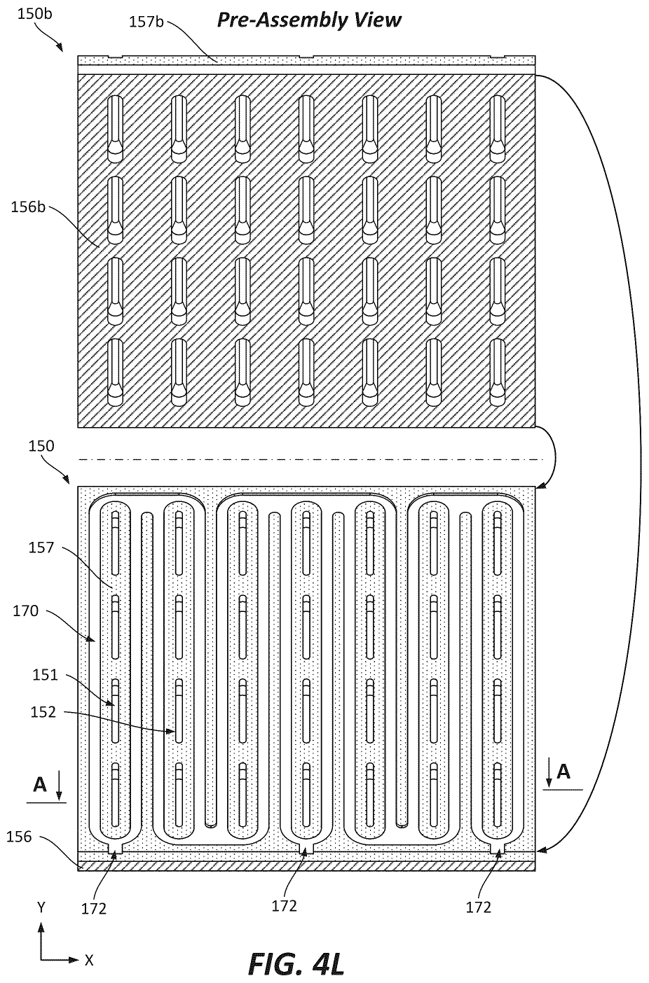

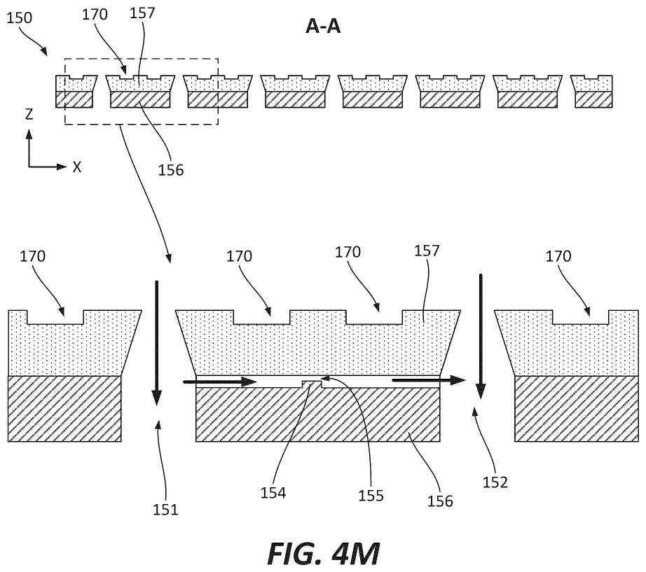

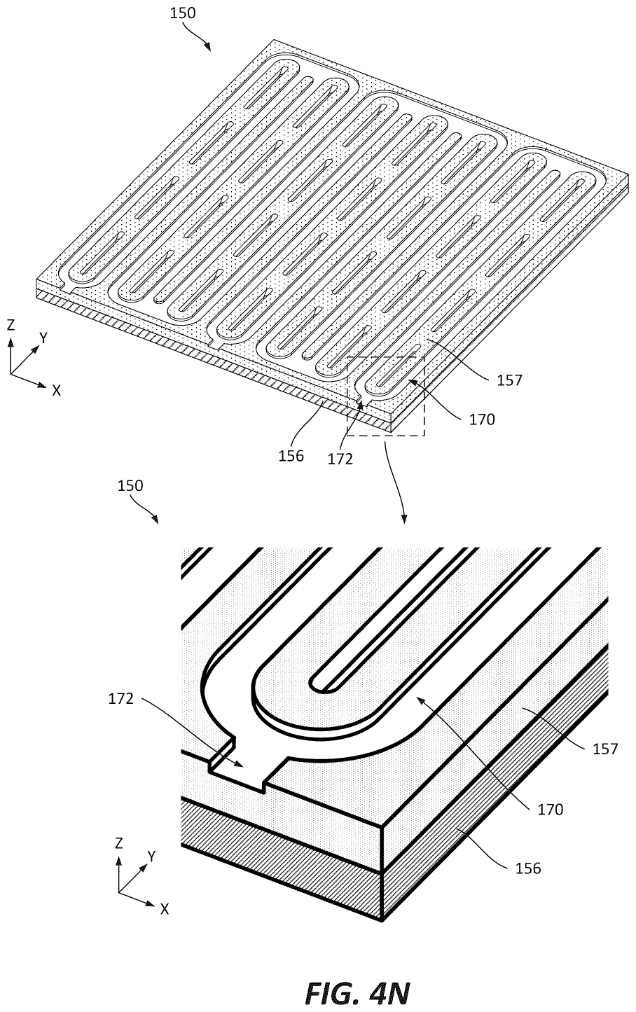

Referring to FIGS. 4A and 4B, in some examples, a processing stack 130 comprises two end gaskets 140 and a processing unit 150 positioned between the two end gaskets 140. When the processing stack 130 has only one processing unit 150, this processing unit 150 directly interfaces both end gaskets 140, e.g., as shown in FIG. 4A. Alternatively, when additional processing units 150 are present in the processing stack 130 (e.g., as shown in FIG. 4B), each additional processing unit 150 has an intermediate gasket 145, corresponding to this additional processing unit 150. Specifically, when the processing stack 130 has two processing units 150, one intermediate gasket 145 is present and is positioned between these processing units 150 (directly interfacing and sealed against each of the processing units 150). One of the end gaskets 140 is positioned and sealed against one of these processing units 150, while the other end gasket 140 is positioned and sealed against the other one of these processing units 150.

In some examples, end gaskets 140 and/or intermediate gasket 145 are formed by casting using elastomers such as silicone rubber. Alternatively, two components that need to be sealed relative to each other can be adhesively bonded to form a seal. For example, a double-sided adhesive tape can be used to bond layers of the processing stack. The double-sided adhesive tape or other bonding materials may be also referred to as sealing gaskets formed in situ. The gaskets and bonding are biocompatible with various types of cell media to prevent a negative impact on the sample viability.

The difference between the end gaskets 140 and intermediate gaskets 145 is the number of gasket slots. Specifically, each end gasket 140 is configured to seal either inlet slots 151 or outlet slots 152 in the processing unit 150, which interfaces this end gasket 140. For example, FIG. 4A illustrates a processing unit 150 positioned between a top-end gasket 140 and a bottom-end gasket 140. In this example, the top-end gasket 140 seals the outlet slot 152 in processing unit 150, while the bottom-end gasket 140 seals the inlet slot 151 in processing unit 150. This approach allows the use of inlet slots 151 and outlet slots 152 that are through holes extending between opposite sides of the processing unit 150 (e.g., for ease of manufacturing and additional functionality). For example, the same type of processing unit 150 can be used for a single-unit processing stack (shown in FIG. 4A) and a multi-unit processing stack (shown in FIG. 4B). In other words, the end gasket 140 has twice as few slots as the processing unit 150 (at least for a given cross-section).

An intermediate gasket 145 has twice as many slots, which may be referred to as intermediate gasket slots 146, as the end gasket 140, referred to FIGS. 4C and 4D. Instead of sealing the slots in the processing unit 150, the intermediate gasket 145 provides fluidic coupling between the inlet slots 151 and, separately, between the outlet slots 152 of the two processing units 150, positioned on the opposite sides of the intermediate gasket 145 (e.g., as shown in FIG. 4B). The intermediate gasket 145 is effectively configured to split and combine the media as the media passed through the processing stack 130. In the example shown in FIG. 4B, the volumetric flow rate of the media through each end gasket slot 142 is twice less than through each intermediate gasket slot 146.

To ensure the flow of media from the pre-processing media reservoir 120 into the processing stack 130, the first one of the two end gaskets 140 is positioned between and sealed against each of the processing unit 150 and the pre-processing media reservoir 120 such that each gasket slots 142 extends perpendicular to the delivery slot 124, e.g., as schematically shown in FIG. 4E. As such, the media is distributed from the delivery slot 124 into gasket slots 142, which then provides fluidic communication to the set of inlet slots 151 positioned of the processing unit 150 positioned on the other side of the end gasket 140. As schematically shown in FIG. 4E, the delivery slot 124 distributes the media along the X-axis, while the media flows from the pre-processing media reservoir 120 into the end gasket 140 or, more specifically, into the set of gasket slots 142 along the Z-axis. The set of gasket slots 142 distributes the media along the Y-axis, while the media flows from the set of gasket slots 142 into the set of inlet slots 151 of the processing unit 150 along the Z-axis.

Further referring to FIGS. 4A, 4B, and 4F, the set of inlet slots 151 is aligned with the set of gasket slots 142 in the first one (top one) of the two end gaskets 140. The set of inlet slots 151 does not overlap with the set of gasket slots 142 in the second one (bottom one) of the two end gaskets 140. The second processing slot 152 does not overlap with any slot in the set of gasket slots 142 in the first one (top one) of the two end gaskets 140 and is sealed by the first one of the two end gaskets 140. The second processing slot 152 is aligned with one slot in the set of gasket slots 142 in the second one (bottom one) of the two end gaskets 140.

Referring to FIG. 4A, in some examples, the processing stack 130 comprises of a processing unit 150 that has the set of inlet slots 151 and the set of outlet slots 152 both positioned on the same side (e.g., on the top side) of the processing unit 150 and outlet slots. This eliminates the need to seal the different types of slots on different sides of the processing unit 150.

Referring to FIG. 4B, in some examples, a processing stack 130 comprises two different types of processing unit 150. One type of processing unit 150 (e.g., positioned on the top) has only inlet slots 151 only on the upper side of the unit (facing away from the other processing unit). The other type of processing unit 150 has only outlet slots on the bottom side of the unit. This eliminates the need to seal outlet slots on the top of the stack and the inlet slots on the bottom of the stack.

While FIG. 4B illustrates a processing stack 130 in which processing units 150 are configured to operate in parallel, in some examples, the processing units 150 may be arranged to operate in series. For example, cell media processed in one processing unit 150 may be then directed into another processing unit 150 for further processing (and one or more additional processing units 150) as needed. Different processing units 150 in such in-series configuration may have different gap sizes thereby subjecting the cell media to different compression levels.

FIGS. 4G is a perspective view of another example of a processing stack 130 comprising seven processing units 150 stacked between the two end gaskets 140 with an intermediate gasket 145 positioned between each adjacent pair of processing units 150. Each end gasket 140 and intermediate gasket 145 comprises two alignment protrusions 147, extending from opposite sides. During the assembly of the processing stack 130, the alignment protrusions 147 may be slid on the alignment fixture (e.g., two parallel pins) which ensures the alignment of the end gaskets 140 and intermediate gaskets 145 relative to each other and each processing unit 150. FIG. 4H is a bottom perspective view of the end gasket 140 in the processing stack of FIG. 4G.

### Additional Examples of Gaskets

FIG. 4I is a cross-sectional schematic side view of a processing stack 130 with two processing units (i.e., a processing unit 150 and a second processing unit 150b). Each processing unit comprises a silicon base (forming a ridge) and a glass cover attached to the silicon base and comprising a seal channel housing an intermediate gasket, in accordance with some examples. Specifically, the processing unit 150 comprises a silicon base 156 and a glass cover 157, while the second processing unit 150b comprises a second silicon base 156b and a glass cover 157b. The processing stack 130 also comprises an intermediate gasket 145 providing a seal between the glass cover 157 and the second silicon base 156b. Similarly, the processing stack 130 may comprise a second intermediate gasket 145b for providing a seal between the second glass cover 157b and another structure positioned above the second glass cover 157b (e.g., another silicon base, pre-processing media reservoir, end plate, etc.). As such, these intermediate gaskets 145 may be used as end gaskets.

FIGS. 4J and 4K are side and top views of the processing stack 130 in FIG. 41. Specifically, these figures illustrate intermediate gaskets 145 protruding to an edge of the processing stack 130 or, more specifically, to the corresponding edge of the silicon base/glass cover (e.g., the bottom edge in the view of FIG. 4K). This “extension to the edge” is an artifact of how these intermediate gaskets 145 are formed. For example, each intermediate gasket 145 may be formed by casting silicone rubber and compressed between the glass cover and a corresponding second silicon base (e.g., the glass cover 157 and the second silicon base 156b in FIG. 4I). It should be noted that each processing unit 150 may be first assembled, e.g., by anodic bonding each glass cover to a corresponding silicon base such as the glass cover 157 to the silicon base 156 and, separately, the second glass cover 157b to the second silicon base 156b. The pre-bonded processing units 150 are then stacked and each intermediate gasket 145 may be in-situ formed/cast between each glass cover to a corresponding silicon base such as the glass cover 157 and the second silicon base 156b.

When stacked, the glass cover 157 comprises a planar surface, which directly interfaces with the second silicon base 156b. This direct interface controls the distribution of the gasket material while forming the intermediate gasket 145. For example, FIG. 4L is a schematic view of two processing units (i.e., the processing unit 150 and the second processing unit 150b) prior to forming a stack and filling a seal channel 170 (with a gasket material), in accordance with some examples. The seal channel 170 is recessed from the planar surface of the glass cover 157 and shaped around the set of inlet slots 151 and set of outlet slots 152. The seal channel 170 comprises a set of seal channel openings 172 that are used to fill the gasket material into the seal channel 170 when the glass cover 157 is stacked with and directly interfacing the second silicon base 156b.

Specifically, the seal channel 170 extends to the edge of the glass cover 157 forming a set of seal channel openings 172. The set of seal channel openings 172 and the shape of seal channel 170 are specifically configured positioning to ensure that seal channel 170 is fully filled with the gasket material. The intermediate gasket 145 is formed in-situ by flowing a gasket material through the set of seal channel openings 172 to fill the seal channel 170 with the gasket material, while the planar surface of the glass cover 157 directly interfaces the second silicon base, and curing the gas material inside the seal channel 170.

FIG. 4M is a cross-sectional view of a processing unit 150 illustrating various features of the silicon base 156 and the glass cover 157, such as a seal channel 170 in the glass cover 157. In some examples, the depth of the seal channel 170 (in the Z-direction) is between 10-50% of the thickness of the glass cover 157 or, more specifically, 20-40%. FIG. 4N is a perspective view of a processing unit 150 illustrating various features of the silicon base 156 and the glass cover 157.

### Examples of Ridges in Processing Units

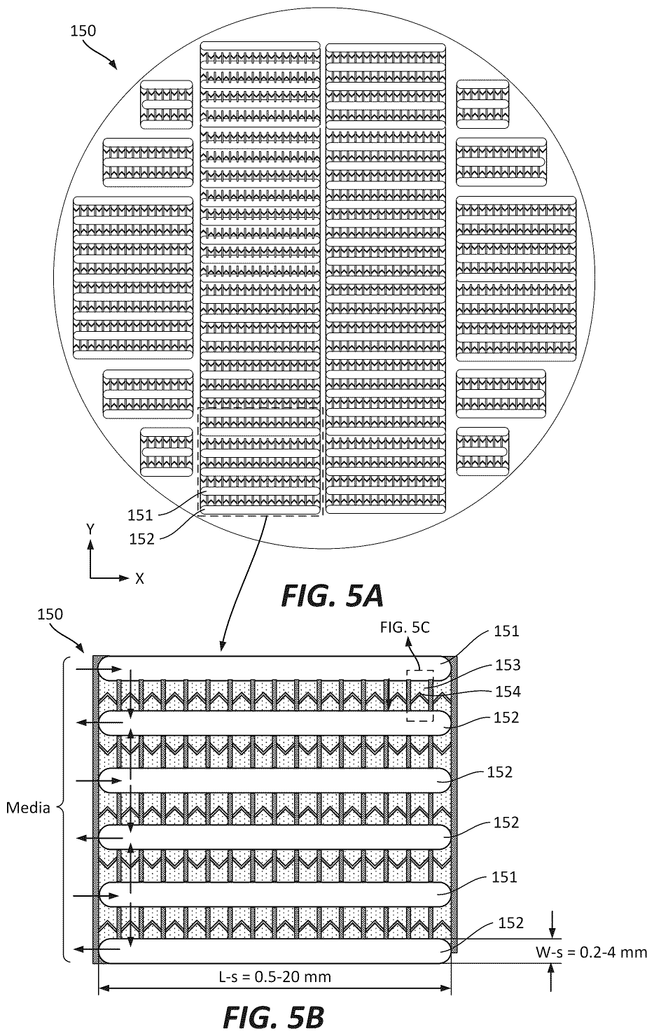

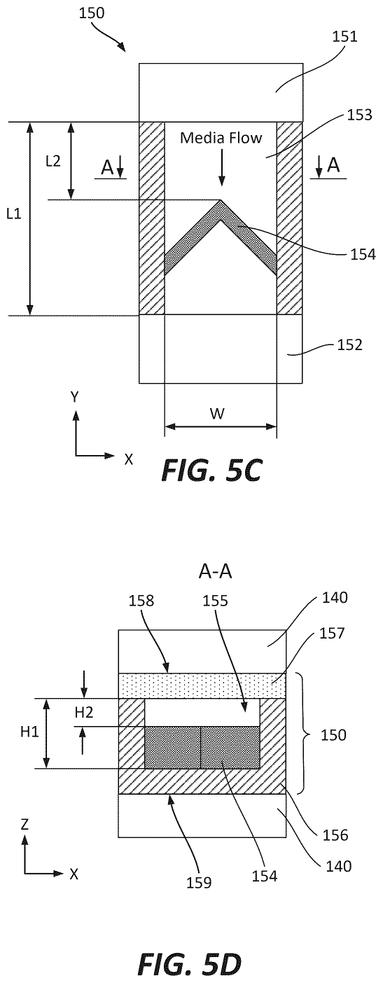

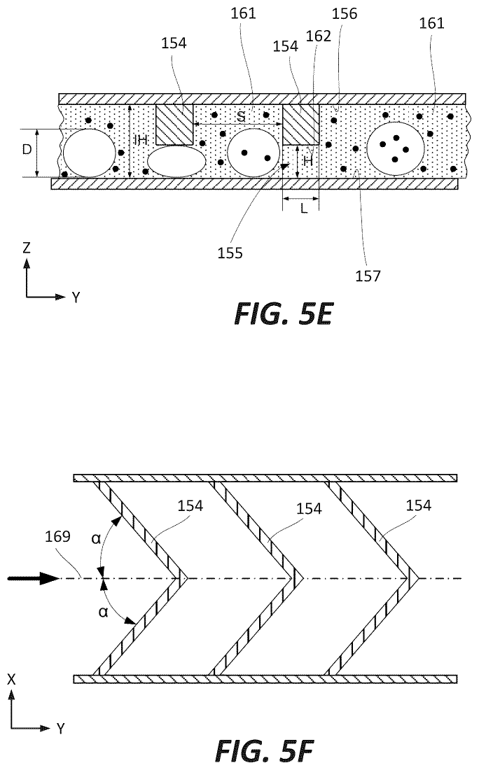

Referring to FIGS. 5A-5D, the processing unit 150 comprises a set of inlet slots 151, a set of outlet slots 152, and a set of processing channels 153 extending between and fluidically coupling the set of inlet slots 151 and the set of outlet slots 152. The processing unit 150 also comprises a set of ridges 154 extending into the set of processing channels 153 and forming a set of gaps 155 within the set of processing channels 153.

In some examples, the processing unit 150 has a circular shape with a diameter of 1.5 inches (38 millimeters) to 2.5 inches (64 millimeters), e.g., 2.0 inches (50 millimeters). For example, a 2-inch wafer may be used for fabricating the processing unit 150. In some examples, the processing unit 150 has a rectangular shape to ensure a larger number of processing channels per unit area. In some examples, the dimensions of rectangular processing units are 2×2 inches, 1×2 inches, 1.5×2 inches, 2×3 inches, and 3×3 inches.

Referring to FIG. 5B, in some examples, one slot of the set of inlet slots 151 extends between the first one of the set of outlet slots 152 and the second one of the set of outlet slots 152. A first subset of the set of processing channels 153 extends between the one slot of the set of inlet slots 151 and the first one of the set of outlet slots 152. A second subset of the set of processing channels 153 extends between the one slot of the set of inlet slots 151 and the second one of the set of outlet slots 152. In other words, the same inlet slot 151 can supply two subsets of processing channels 153, positioned on both sides of this inlet slot 151. Similarly, the same outer slot 152 can receive media from two subsets of processing channels 153, positioned on both sides of this outlet slot 152. This inlet/outlet slot-sharing helps to increase the density of the processing channels 153 in a given area and, therefore, the throughput of each processing unit 150, which is further described below. In some examples, the width (W-s) of the inlet slots 151/outlet slots 152 is between 0.2-4 millimeters or, more specifically, between 0.5-2 millimeters. In the same or other examples, the length (L-s) of the inlet slots 151/outlet slots 152 is between 0.5-20 millimeters or, more specifically, between 1-10 millimeters.

Referring to FIG. 5D, in some examples, the processing unit 150 comprises a silicon base 156 and a glass cover 157 attached to the silicon base 156 and forming the set of processing channels 153 with the silicon base 156. The set of ridges 154 is a part of the silicon base 156. For example, silicon wafers can be lithographically etched to achieve specific ridge and gap configurations.

Referring to FIG. 5D, in some examples, the processing unit 150 comprises a processing-unit first surface 158 and a processing-unit second surface 159. For example, the processing-unit first surface 158 may be formed by the glass cover 157 and may directly interface with the first one of the two end gaskets 140. The processing-unit second surface 159 formed by the glass cover 157 may directly interface the second one of the two end gaskets 140 or the intermediate gasket 145. Each slot in the set of inlet slots 151 and the set of outlet slots 152 extends between the processing-unit first surface 158 and the processing-unit second surface 159.

Referring to FIGS. 5C and 5D, in some examples, each of the set of processing channels 153 has 1 to 5 ridges of the set of ridges 154. For example, FIG. 5D illustrates a single ridge positioned in the processing channel 153.

Referring to FIG. 5C, in some examples, each ridge of the set of ridges 154 has a chevron shape with a leading chevron edge facing a corresponding one of the set of inlet slots 151. Without being restricted to any particular theory, it is believed that the chevron shape helps with more efficient cell compression for a given flow rate and delays clogging.

Referring to FIG. 5C, in some examples, each of the set of processing channels 153 has a length (L1), defined by a distance between a corresponding one of the set of inlet slots 151 and a corresponding one of the set of outlet slots 152, of 1 millimeter to 10 millimeters. While the larger length may be beneficial to stabilize the flow within the processing channels 153 (as the media flows in these processing channels 153 from the inlet slots 151), the larger length reduces the number of processing channels 153, which can be positioned in a given area of the processing unit 150 and which may be referred to as a channel density. As a reference, the area of the 2-inch wafer is about 20 centimeters square.

Referring to FIG. 5C, in some examples, the width (W) of each processing channel 153 can be 0.01-10 millimeters or, more specifically, 0.3-2 millimeters.

Referring to FIG. 5D, in some examples, the channel height (H1) of each processing channel 153 is 5-50 micrometers or, more specifically, 10-30 micrometers. The channel width (W) of each processing channel 153 is 10-10000 micrometers, more specifically 100-1000 micrometers. The channel length L1 of each processing channel is 10-10000 micrometers, and more specifically 200-1000 micrometers. In some examples, the channel length L1 is the same for all channels. In some examples, the channel length L1 varies to compensate for pressure loss in the media distribution system. The gap height (H2) may be 2-20 micrometers or, more specifically, 5-12 micrometers. The gap height (H2) is typically selected based on the cell size and can represent 10-90% of the average cell size or more specifically, 30-60% of the average cell size.

It should be noted that the set of ridges 154 provides a fluidic block for the media when the media is initially introduced into the high-throughput high-volume mechanoporation unit 100. Specifically, the media experiences static pressure when added to the media cavity 122 and flows through the delivery slot 124 and the set of gasket slots 142 into the set of inlet slots 151 and eventually into the set of processing channels 153. The set of ridges 154, which defines the set of gaps 155, characterized by gap height (H2), blocks the further flow of the media, e.g., into the second enclosure portion 190. In other words, the design of various pathways, in particular the set of processing channels 153, and the set of gaps 155 are specifically designed in such a way (taking into account the properties of the media, e.g., its surface tension, viscosity) to bring the media to the set of ridges 154 and make the media ready for immediate processing (i.e., compression of the cells in the media within the set of gaps 155).

Referring to FIG. 5E, each gap 155 is identified with a corresponding height, labeled as “H”. The gap height is selected such that cells are compressed to pass through gap 155. In other words, the gap height is smaller than the cell size (H<D). It should be noted that FIG. 5E illustrates an example in which the cross-sectional profile of ridge 154 is rectangular. However, other shapes of the profile are also within the scope, e.g., cylindrical, trapezoidal, or triangular. In some embodiments, the plurality of compressive surfaces may be orthogonal.

Another characterization, at least of rectangular ridges 154, is the length of ridge surface 162 in the X direction (identified as “L”), which may be also referred to as the ridge thickness. In some examples, the ridge surface length is between about 1 micrometer and 100 microns or, more specifically between about 20 micrometers and 50 micrometers.

This length, together with the linear flow rate, defines the period during which a cell is compressed by the ridge. In some examples, ridge surface 162 is parallel to the surface of the glass cover 157. In other words, gap 155 is defined by two parallel surfaces. These parallel compressive surfaces allow for uniform compression for the entire cell. Additionally, the compression surfaces can be converging and/or diverging. It should be noted that in addition to cell compression, ridges 154 also produce hydrodynamic mixing within the cell media.

Referring to FIG. 5E, the gap 155 is selected based on the cell size, compression needed, and other characteristics of mechanoporation. In some examples, the gap height (H) is between 1 micrometer and 20 micrometers, or between 10 micrometers and 100 micrometers or, more specifically, between 3 micrometers and 8 micrometers. Furthermore, the gap height (H) may be also defined relative to the cell size (D), which is defined as the average largest cross-sectional dimension of cells. More specifically, the ratio of the gap height to the cell size (H/D) defines cell compression. In some examples, this H/D ratio is between 25% and 75% or, more specifically, between 30% and 60%. Internal channel height (IH) defines the flow speed between ridges and the time that the cell spends between consecutive compressions. In some examples, the IH is between about 2 micrometers and 100 micrometers or more specifically between 5 micrometers and 10 micrometers or between 10 micrometers and 15 micrometers. In some examples, IH is between about 10 micrometers and 1 mm or more specifically between 50 micrometers and 100 micrometers.

The gap height (H) may be the same for all ridges 154 in the same channel. Alternatively and with reference to FIG. 5E, the gap height (H) may be different for different ridges 154. For example, the gap height decreases along the direction of the flow thereby subjecting the cells to higher compression as the cells flow through mechanoporation unit 100. In some examples, when larger cells are compressed in larger gaps, these cells may retain a flattened (pancake-like) shape and can then pass through smaller gaps, again without being removed from the flow. This feature may be referred to as a staged-compression. Furthermore, the smaller gaps may start processing smaller cells, if present (e.g., in a diverse population). Thus, using gap size varying along the channel can improve convective intracellular delivery to heterogeneous cell populations. Furthermore, such channels with varying compression gaps can be used to improve cell sorting by reducing the influence of cell size heterogeneity.

Intracellular delivery is controlled by cell compression rate, which is a rate of volume loss by cells in order to pass through the gap formed by a ridge. The cell compression rate can be determined by the flow rate, ridge geometry, a ratio of the gap height to the cell size, ridge width, ridge angle, and compressive surface coating. Furthermore, it has been found that the volume loss (Vloss) increases with the increase in the cell compression rate. Various processing and device characteristics may be specifically selected to achieve the desired cell compression rate. In some examples, ridges 154 are oriented at an angle (α) between 0 degrees to 90 degrees relative to a central flow axis 169 in each channel as, e.g., is schematically shown in FIG. 5F. More specifically, the ridge angle (α) is between 10 degrees to 30 degrees, or between 30 degrees and 90 degrees, more specifically about 45 degrees. In some examples, ridges 154 form a chevron as, e.g., is shown in FIG. 5F. Alternatively, ridges 154 are straight. In some examples, ridges 154 are curved. Furthermore, in some examples, each ridge 154 can extend both sidewalls or form a sidewall gap with at least one of the sidewalls. This sidewall gap may be referred to as a gutter and may receive uncompressible cells (e.g., pushed along ridges 154 into the sidewall gap) thereby reducing the risk of channel clogging.

It should be noted that the cross-sectional shape of ridges 154 (shown in FIG. 5E and 5D) defines the cell compression profile. In some examples, the shape is rectangular (shown in FIG. 5D), trapezoidal, or triangular. In some examples, ridge surface 162 forming compression gap 155 is substantially flat (e.g., parallel to the surface of the glass cover 157) or tilted forming a gap varying along the X-axis. In some examples, ridge surface 162 is nearly cylindrical.

Referring to FIG. 5E, spaces within the channel between adjacent ridges 154 and after the last ridge and outlet may be referred to as recovery spaces 161. The length of recovery spaces 161 (in the X direction) between two adjacent ridges may be referred to as ridge spacing (S). The ridge spacing depends on the flow rate, cell characteristics, levels of the previous compression, and such. In some examples, the ridge spacing is between 1 micrometer and 100 micrometers or between 50 micrometers and 10,000 microns such as between 200 micrometers and 500 micrometers. The volume of each recovery space 161 and the flow rate determines the average recovery time, i.e., the time that the cells spend in recovery space 161 before being subjected to another compression. It has been found that volume gain (Vgain) is increased when the recovery time is increased. The recovery time can be increased by increasing the length of recovery spaces 161.

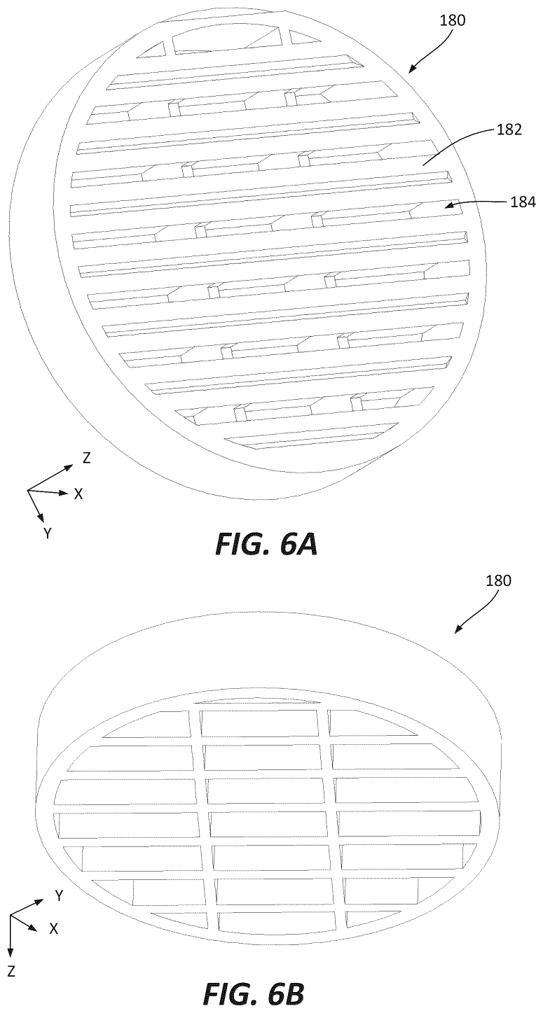

Referring to FIGS. 6A-6D, in some examples, a stack support 180 comprises a set of rib 182 separated by a set of openings 184. Specifically, the stack support 180 interfaces the second one of the two end gaskets 140 with the set of rib 182 provides support to the processing stack 130. It should be noted that a pressure differential across the processing stack 130 can be as much as 100 psi (689 kPA). Specifically, the set of openings 184 is aligned with the set of gasket slots 142 in the second one of the two end gaskets 140.

Referring to FIG. 6D, in some examples, the stack support 180 comprises a stack-facing surface 181 that interfaces the second one of the two end gaskets 140. The set of openings 184 taper away from the stack-facing surface 181.

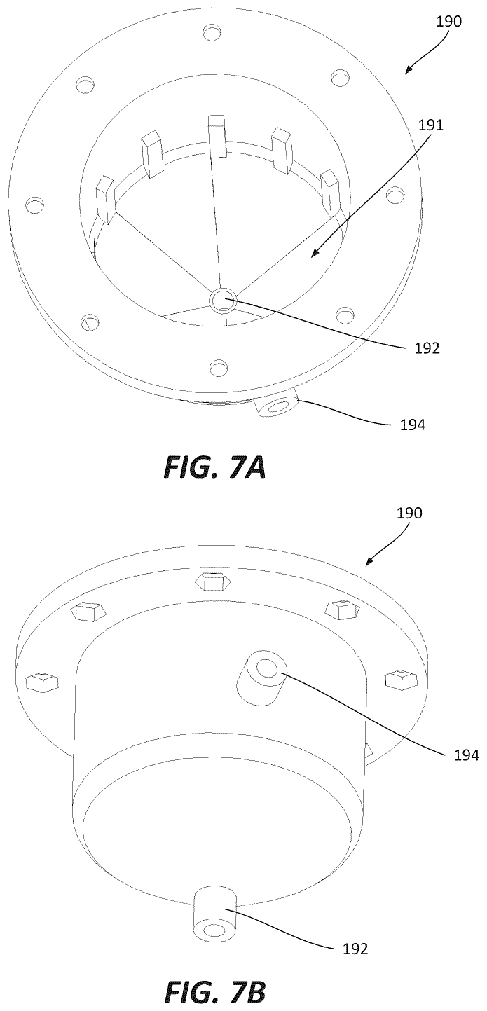

Referring to FIGS. 7A-7B, in some examples, a second enclosure portion 190 comprises a media outlet 192 and a gas outlet 194 and is connected to the 110. The second enclosure portion 190 is attached to the first enclosure portion 110. The second enclosure portion 190 also comprised a cavity 191 fluidically coupled to the set of outlet slots 152 and used for collecting the cell media after passing through the processing stack 130.

### System Examples

FIG. 8A illustrates a block diagram of a high-throughput high-volume mechanoporation apparatus 800 that comprises a high-throughput high-volume mechanoporation unit 100, various examples of which are described above. Specifically, the high-throughput high-volume mechanoporation unit 100 can be a disposable component that is replaced for each operation of the high-throughput high-volume mechanoporation apparatus 800 (e.g., a new batch of cell media supplied to the high-throughput high-volume mechanoporation apparatus 800). As noted above, the high-throughput high-volume mechanoporation unit 100 and, as a result, the high-throughput high-volume mechanoporation apparatus 800 may operate in a batch manner determined by the throughput of the high-throughput high-volume mechanoporation unit 100 (e.g., limited by clogging as described above). Once a batch of cell media is processed, the high-throughput high-volume mechanoporation unit 100 is removed from the high-throughput high-volume mechanoporation apparatus 800 and replaced with another high-throughput high-volume mechanoporation unit 100. These two high-throughput high-volume mechanoporation units 100 may have the same design (e.g., the volume of the media cavity 122, the number and design of processing channels, and the like) or different (e.g., different units may be designed to process different types of cells by using different designs of processing channels such as different gap sizes).

Returning to FIG. 8A, the high-throughput high-volume mechanoporation apparatus 800 may comprise an on-board air source 812, such as an air compressor. The onboard air source 812 is optional, and the high-throughput high-volume mechanoporation apparatus 800 can be connected to an external air supply (e.g., a high-pressure line, an external compressor, etc.). The high-throughput high-volume mechanoporation apparatus 800 may comprise a pressure regulator 814, e.g., to drop the incoming pressure to a desired level, such as 1-150 psi or, more specifically, 5-40 psi. The high-throughput high-volume mechanoporation apparatus 800 may comprise an upstream ballast 816, e.g., to store the pressurized air. Specifically, the upstream ballast 816 is used to maintain the substantially constant air pressure during the entire processing cycle to ensure that the entire cell processing is performed at uniform conditions. In some examples, the volume of the upstream ballast 816 is 0.01-100 liters or, more specifically, 0.1-2 liters. The high-throughput high-volume mechanoporation apparatus 800 may comprise an upstream valve 818 that initiates and stops the processing cycle as further described below. Various types of upstream valve 818 are within the scope. The upstream valve 818 and the onboard air source 812 (if one is present) may be controlled by controller 890.

Returning to FIG. 8A, the high-throughput high-volume mechanoporation apparatus 800 or, more specifically, the high-throughput high-volume mechanoporation unit 100 may comprise an internal upstream filter 822, which ensures that the contaminants (e.g., from pressurized air received from the upstream valve 818) do not reach other internal components of the high-throughput high-volume mechanoporation unit 100 or, more specifically, do not reach the cell media after the cell media is supplied into the high-throughput high-volume mechanoporation unit 100. Various types of internal upstream filter 822 are within the scope.

Returning to FIG. 8A, in some examples, the high-throughput high-volume mechanoporation unit 100 comprises an internal ballast 826, which is used to minimize the effects of the airflow restrictions through the internal upstream filter 822. The internal ballast 826 may be used together with the upstream ballast 816. In some examples, the volume of the internal ballast 826 is 0.01-10 liters or, more specifically, 0.1-1 liter. The volume of the internal ballast 826 may be smaller than the volume of the upstream ballast 816, e.g., at least twice smaller, at least 5 times smaller, at least 10 times smaller (to reduce the size and cost of the high-throughput high-volume mechanoporation unit 100, which may be a disposable component). When the internal ballast 826 is isolated from the media cavity 122 from the media cavity 122 by an internal valve 828 (e.g., a pinch valve), controlled by the controller 890. In these examples, the internal valve 828 is used to trigger the operation (i.e., pushing the cell media from the media cavity 122 into and through the processing unit 150, and eventually into a second enclosure portion 190 (all of which are described above). In some examples, the filter 822, internal ballast 826, valve 828, mechanoporation unit 100, and connecting tubbing are disposable, e.g. they are replaced after sample processing is complete.

Returning to FIG. 8A, in some examples, the high-throughput high-volume mechanoporation unit 100 comprises an internal downstream filter 829 that prevents contaminants from getting into the high-throughput high-volume mechanoporation unit 100 through the gas outlet 194. Various types of internal downstream filter 829 are within the scope.

In addition to controlling various valves (e.g., an upstream valve 818, an internal valve 828), the controller 890 is also configured to receive inputs from various sensors of the high-throughput high-volume mechanoporation apparatus 800. For example, the high-throughput high-volume mechanoporation apparatus 800 may comprise an inlet pressure sensor 832 for measuring the pressure at a gas inlet 114 of the high-throughput high-volume mechanoporation unit 100. Similarly, an outlet pressure sensor 836 for measuring the pressure at a gas outlet 194 of the high-throughput high-volume mechanoporation unit 100. In some examples, a media-cavity pressure sensor 834 may be used to measure gas pressure inside the media cavity 122, e.g., over the surface of the cell media.

In some examples, the high-throughput high-volume mechanoporation apparatus 800 may be equipped with a flow sensor 838 for controlling the flow rate of air through the gas outlet 194. Specifically, when the process is activated and the cell media is initially pressurized into the processing unit 150, the flow rate through the gas outlet 194 is equivalent to the flow rate of the cell media through the channel (e.g., between 0.1 ml/second and 1000 ml/second). This “liquid processing” flowrate depends on the number of processing channels and the flow rate through each channel (determined by the properties of the cell media (e.g., viscosity, surface tension, etc.) and design of each channel (e.g., cross-sectional area, length, gap size, number of ridges, etc.). Once the cell media is mostly displaced from the media cavity 122, the clean air (used to pressurize the cell media) will reach one or more of the processing channels and escape through these channels into the second enclosure portion 190. At this point, the airflow rate will increase rapidly above the “liquid processing” flow rate, and controller 890 will use this information to shut down one or both of the upstream valve 818 and the internal valve 828. Additional aspects of the operation of the high-throughput high-volume mechanoporation apparatus 800 will now be described with reference to FIG. 9.

FIGS. 8B and 8C illustrate another example of the high-throughput high-volume mechanoporation unit 100 that comprises a media isolator 118. The media isolator 118 is used to isolate the air (e.g., coming from an onboard air source 812 or some external sources and used to displace the cell media) from the cell media. Specifically, the air applies the pressure on the media isolator 118, which changes the shape and presses onto the cell media, pushing the media from the media cavity 122 through the processing stack 130 into a media collector (e.g., in the second enclosure portion 190) as schematically shown in FIGS. 8B and 8C. In this example, the air does not come in direct contact with the cell media and, therefore, does not need to be cleaned. In other words, the high-throughput high-volume mechanoporation unit 100 may be used without an internal upstream filter 822 or any other such filters.

Specifically, the media isolator 118 may deflect within the media cavity 122 up until the media isolator 118 conforms to the wall of the media cavity 122 and the processing stack 130. The cell media is displaced through the processing stack 130. In this example, a flow sensor may not be needed to stop processing since the media isolator 118 prevents the air from going through the processing stack 130. Flow of air through the processing stack 130 may negatively impact the process outcome such as by drying the media and by promoting the formation of foam. A flow sensor may be used at gas outlet 194 to determine when the displacement of gas through gas outlet 194 is zero and the process is completed.

Furthermore, it should be noted that the example in FIGS. 8A and 8B may produce better processing yield by pushing more cell media through the processing stack 130 before ending the cycle. In other words, less cell media is left in the media cavity 122 at the end of the cycle (the “dead volume” in the media cavity 122 is minimized). Without the media isolator 118, the air may find its way to the first processing channel prematurely.

Referring to FIGS. 8B and 8C, the media isolator 118 may be a planar structure (prior to supplying compressed air into the media cavity 122). The edges of the 118 may be supported and sealed on the walls of the media cavity 122. The media isolator 118 may be also referred to as an elastic media displacement unit, an elastic media isolator, an elastic diaphragm, a membrane, and the like. The media isolator 118 changes its shape (e.g., stretches to conform to the walls of the media cavity 122 and the processing stack 130). In some examples, the media isolator 118 may be formed from various flexible materials, such as silicone, rubber, and other materials. It should be noted that the media isolator 118 should be sufficiently flexible to ensure that the media isolator 118 can stretch and deform to its “end of cycle” shape with a set pressure level. The pressure required to stretch and deform the media isolator 118 should be sufficiently small compared to the sample processing pressure, e.g. less than 0.1 PSI, less than 0.5 PSI, less than 1 PSI, less than 10 PSI. In some examples, the media isolator 118 stretches by at least about 25%, at least about 50%, and at least about 100% on average. The pressure required to stretch media isolator 118 needs to be added to the pressure needed to force the cell media through the processing stack 130 for efficient processing. Furthermore, the media isolator 118 should have a relatively small weight and the moment of inertia to avoid any substantial delays during the medial isolator 118 stretching related to its acceleration from the initial resting position.

FIGS. 8D and 8E illustrate yet another example of the high-throughput high-volume mechanoporation unit 100 that comprises a media isolator 118, formed as a bulb. This example of the media isolator 118 may be sealed onto the gas inlet 114, which may be easier than sealing against the walls of the media cavity 122.

FIGS. 8F and 8G illustrate yet another example of the high-throughput high-volume mechanoporation unit 100 that comprises a media isolator 118, which maintains a pre-folded shape before applying the air pressure/force onto the media isolator 118. When the air is supplied on the air-side of the media isolator 118, the media isolator 118 proceeds to unfold and change its shape (e.g., to conform to the sidewalls of the media cavity 122 and the processing stack 130). In this example, the media isolator 118 may not need to stretch as much as in FIGS. 8B and 8C (or FIGS. 8D and 8E). In some specific examples, the media isolator 118 does not stretch at all and all of the shape changes are achieved through unfolding.

FIGS. 8H and 8I illustrate yet another example of the high-throughput high-volume mechanoporation unit 100 that comprises a media isolator 118 that is operable to change its shape by deflection (flip-flopping between two different sides of the media cavity 122). This design may be used to reduce the stretching requirements on the media isolator 118. For example, a thin piece of metal or a relatively rigid plastic or rubber can be used in this example.

FIGS. 8J and 8K illustrate yet another example of the high-throughput high-volume mechanoporation unit 100 that comprises a media isolator 118 is folded around a media cavity separator 129 thereby separating the media cavity 122 into multiple sub-cavities. In some examples, each sub-cavity has a separate media isolator 118 such that the high-throughput high-volume mechanoporation unit 100 comprises multiple media isolators 118. Different sub-cavities may be filled from the same port (shown) or different ports (not shown).

FIGS. 8L and 8M illustrate yet another example of the high-throughput high-volume mechanoporation unit 100 that comprises a media isolator 118 slidably and sealably coupled to the sidewalls of the media cavity 122. Initially, the media isolator 118 is positioned proximate to the gas inlet 114 allowing the space between the media isolator 118 and the processing stack 130 to be filled with the cell media. The compressed air is then supplied through the gas inlet 114 and forces the media isolator 118 to slide toward the processing stack 130 thereby displacing the cell media through the processing stack 130. In this example, the media isolator 118 can be a rigid structure that operates like a piston with the media cavity 122 operating like a cylinder.

It should be noted that sealing the media isolator 118 against the media cavity 122 eliminates the need for other seals in the high-throughput high-volume mechanoporation unit 100 such that the media isolator 118 separates the air-pressure side from the media-side of the high-throughput high-volume mechanoporation unit 100.

### Method Examples

FIG. 9 is a process flowchart corresponding to method 900 of operating a high-throughput high-volume mechanoporation apparatus 800, in accordance with some examples. Various examples of the high-throughput high-volume mechanoporation apparatus 800 and of the high-throughput high-volume mechanoporation unit 100 are described above.

The method 900 may commence with (block 902) installing a high-throughput high-volume mechanoporation unit 100 to a high-throughput high-volume mechanoporation apparatus 800. As noted above, the high-throughput high-volume mechanoporation apparatus 800 may be used for batch operations, such that each new batch of cell media is processed using a new high-throughput high-volume mechanoporation unit 100. The new high-throughput high-volume mechanoporation unit 100 may replace a previously used high-throughput high-volume mechanoporation unit 100 (which may have been partially clogged during previous processing). The replacement nature of high-throughput high-volume mechanoporation unit 100 allows repeated operations of the high-throughput high-volume mechanoporation apparatus 800 despite the inherent clogging of processing channels in high-throughput high-volume mechanoporation units 100. Furthermore, the replacement nature of high-throughput high-volume mechanoporation unit 100 allows to tailor characteristics of the high-throughput high-volume mechanoporation unit 100 to specific types of cells and, more generally, cell media (e.g., cell sizes, cell concentration, media, payload, etc.). In some examples, the installation operation (block 902) involves connecting the gas inlet 114 of the new high-throughput high-volume mechanoporation unit 100 to the line extending to the upstream valve 818 (e.g., as described above with reference to FIG. 8A).

Returning to FIG. 9, method 900 may comprise (block 910) delivering a cell media to the pre-processing media reservoir 120. For example, the cell media may be supplied through the media inlet 112 in the first enclosure portion 110. In some examples, delivering media to the pre-processing media reservoir 120 comprises flowing the media, under static pressure from the media, to the set of ridges 154. In other words, the high-throughput high-volume mechanoporation unit 100 may be positioned in such a way that the media inlet 112 is positioned gravitationally higher than the processing unit 150. This operation may be referred to as the pre-processing filling of the high-throughput high-volume mechanoporation unit 100. The set of ridges 154 acts as the fluid blockers since the gaps formed by these ridges are generally smaller than the cell sizes.

Various examples of cell media are within the scope. In some examples, the cell media has a cell density of at least 20×10\hat{ }6 cells per milliliter, at least 40×10\hat{ }6 cells per milliliter, and at least 100×10\hat{ }6 cells per milliliter. The volume of the cell media added to the high-throughput high-volume mechanoporation unit 100 may be substantially the same as the volume of the media cavity 122 (e.g., within 80% of the media cavity volume or even within 90% of the media cavity volume) to ensure that there is limited uncompressed airgap about the media surface.

In some examples, cell media is premixed with one or more reagents when it is fluidically supplied into the media cavity 122. In some examples, cell media and reagents are separately fluidically supplied into the media cavity 122, where cell media and reagents are mixed either passively or actively. In some examples, cell media and reagents are mixed immediately before they are supplied into the media cavity 122. In some examples, cell media and reagents are agitated in the media cavity 122, e.g. using a mechanical or ultrasound agitation, to prevent the gravitational settling of cells and/or reagents and maintain uniform cell and reagent densities through the processing by the processing unit 100. In some examples, the media cavity 122 is maintained at a constant temperature, e.g. 36° C. to 37° C., to improve cell conditions. In some examples, the temperature of the media cavity 122 is reduced, e.g. 5° C. to 20° C., to improve cell survivability.

Method 900 may comprise (block 920) increasing the air pressure in the pre-processing media reservoir 120 over the media to at least 5 psi (34 kPa) or, more specifically, to at least 10 psi (69 kPa) or even at least 20 psi (138 kPa) or, more while monitoring the air flow rate through the gas outlet 194. Specifically, the upstream valve 818 and/or the internal valve 828 may be opened to let the air into the media cavity 122 thereby pushing the cell media into the processing unit 150 and through the processing unit 150 or, more specifically, through the set of gaps 155 formed by the set of ridges 154. As noted above, the airflow rate represents when most of the cell media has been displaced from the media cavity 122 and the air is escaping through the processing unit 150 (one or more channels of the processing unit 150), which corresponds to a rapid increase in the airflow.

Method 900 may comprise (block 930) reducing the air pressure in the pre-processing media reservoir 120 to atmospheric level as soon the air flow rate through the gas outlet 194 increases rapidly. Method 900 may comprise (block 940) removing the media through media outlet 192.

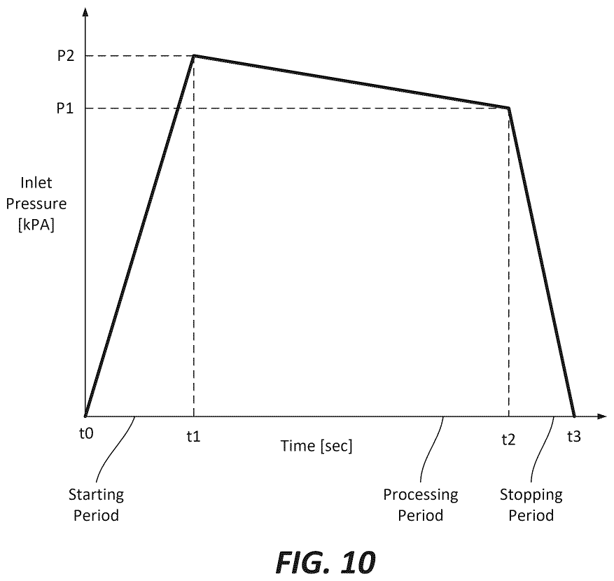

FIG. 10 illustrates a pressure profile at the media inlet 112 as a function of the time (e.g., measured using the inlet pressure sensor 832). The goal is to shorten the starting period (t0-t1) and the stopping period (t2-t3) while maintaining as constant pressure as possible during the processing period (t1-t2). This goal is achieved by various design features described above. In some examples, each of the starting period (t0-t1) and the stopping period (t2-t3) is less than 1 second, less than 0.5 seconds, e.g., 10-500 milliseconds. For purposes of this disclosure, the transition between the starting period (t0-t1) and the processing period (t1-t2) is defined by the pressure maximum. Specifically, when the process starts at t0 (e.g., by opening one or both of the upstream valve 818 and internal valve 828), the air flows from the one or both ballasts until it reaches the maximum, which is slightly less than the initial pressure in the ballasts. The air then pushes the cell media through the processing unit 150 thereby using the air from the ballasts and causing the inlet pressure to slowly increase. The relative volumes of the ballast (Vb) and the media (Vm) determine the amount of pressure dropped during the processing period (t1-t2), e.g., P1×Vb=P2×(Vb+Vm). When most of the cell media has been displaced from the media cavity 122 and the air is escaping through the processing unit 150, the inlet pressure drops rapidly in the stopping period (t2-t3). The speed with which the controller 890 can receive the input and shut down the upstream valve (e.g., the upstream valve 818 and/or the internal valve 828) determines the duration of the stopping period (t2-t3), which can be less than 1 second, less than 0.5 seconds, e.g., 10-500 milliseconds. Shortening the starting period (t0-t1) is important to reduce the amount of cells processed in suboptimal conditions. Shortening the stopping period (t2-t3) is important to reduce cell media foaming and evaporation due to the rapid airflow in the second enclosure portion 190.

### Additional Examples of High-Throughput High-Volume Mechanoporation Units

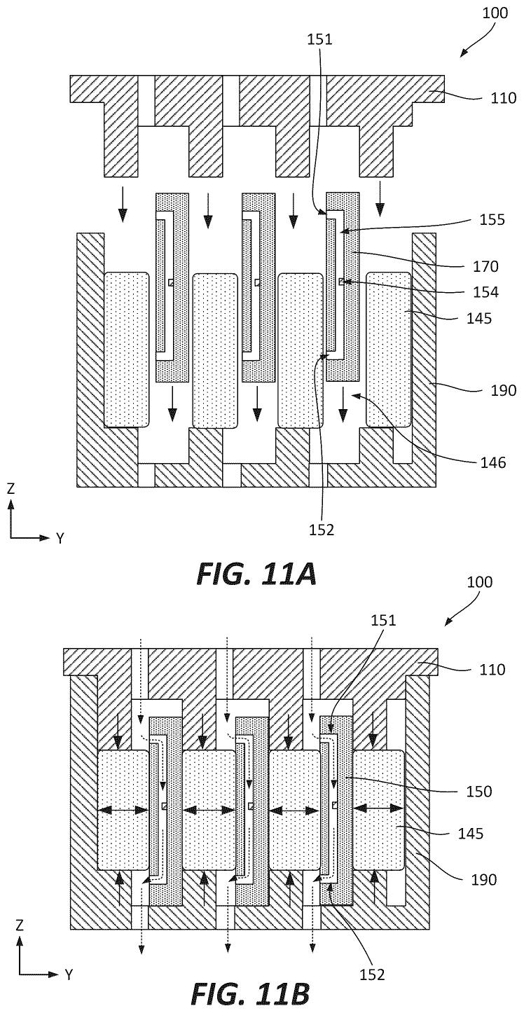

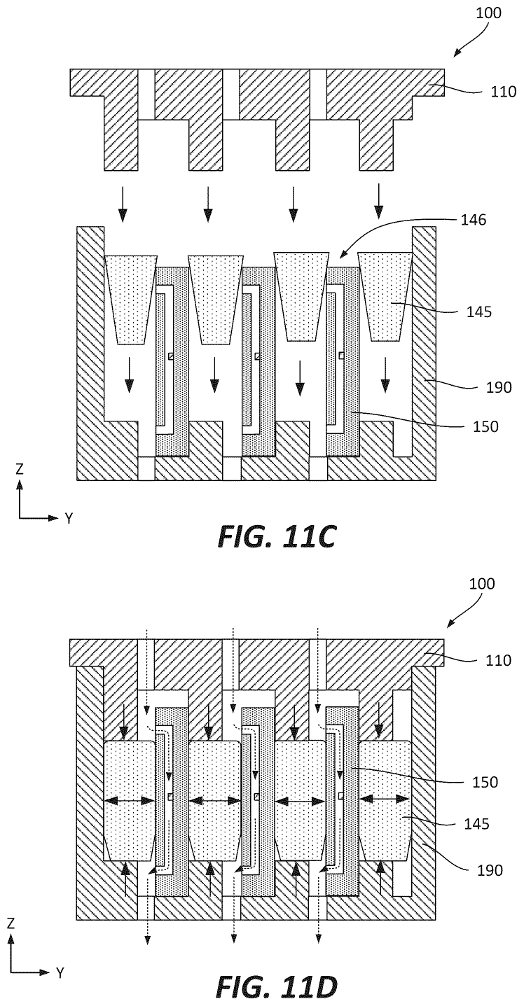

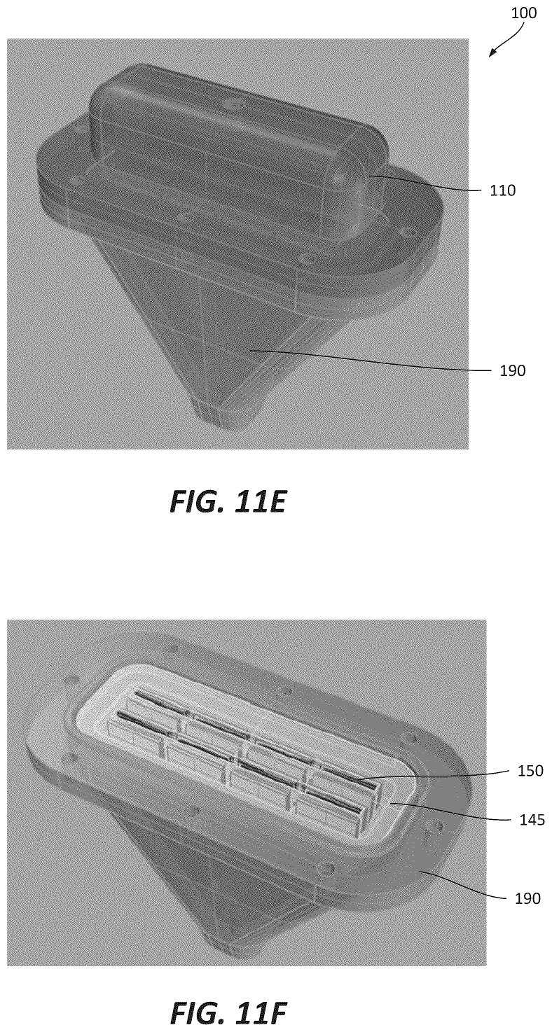

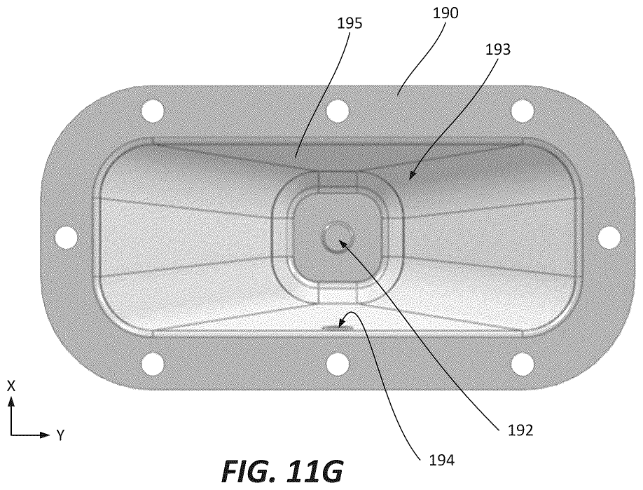

FIGS. 11A and 11B are side cross-sectional views of another example of a high-throughput high-volume mechanoporation unit 100 during and after the unit assembly. The high-throughput high-volume mechanoporation unit 100 comprises a first enclosure portion 110 and a second enclosure portion 190 that enclose a gasket (e.g., an intermediate gasket 145) and a set of processing units 150. Specifically, the intermediate gasket 145 includes intermediate gasket slots 146, each receiving one processing unit 150. Unlike processing units described with reference to FIGS. 4A and 4B, each processing unit 150 in FIGS. 11A and 11B (as well as FIGS. 11C and 11D) comprises a set of inlet slots 151 and a set of outlet slots 152 that are positioned on the same side of the processing unit 150. For example, the base of a processing unit 150 may be a silicon chip with a set of gaps 155 and a set of ridges 154 lithographically formed on the surface. A cover (e.g., a glass sheet) may be positioned over this base to form the enclosed gaps as well as the set of inlet slots 151 and the set of outlet slots 152.

When the processing unit 150 protrudes into the corresponding intermediate gasket slot 146 and when then the intermediate gasket 145 is compressed, the intermediate gasket 145 seals against the portion of the processing unit side that separates the set of inlet slots 151 and the set of outlet slots 152 (e.g., as shown in FIG. 11B). As such, when the cell media is pressurized into the set of inlet slots 151, the intermediate gasket 145 prevents the cell media from bypassing the set of gaps 155 (e.g., from flowing between the processing unit 150 and the intermediate gasket 145).

The intermediate gasket 145 may be compressed using and between the first enclosure portion 110 and the second enclosure portion 190. For example, the compression may be achieved during the assembly of the high-throughput high-volume mechanoporation unit 100, e.g., when the first enclosure portion 110 is attached to the second enclosure portion 190. However, other components may be used for compressing the intermediate gasket 145. It should be noted that the intermediate gasket 145 is compressed without blocking the set of inlet slots 151 and the set of outlet slots 152.

FIGS. 11C and 11D illustrate another example in which the intermediate gasket 145 is tapered. The taper may be used to assist with the sealing of the processing units 150 against the intermediate gasket 145, e.g., when the processing units 150 are inserted into the intermediate gasket slots 146. In some examples, the taper may be provided on the processing units 150 and/or the second enclosure portion 190 (as shown in FIG. 11G) in addition to or instead of the taper on the intermediate gasket 145.

FIG. 11E illustrates an example of a high-throughput high-volume mechanoporation unit 100, in which the first enclosure portion 110 is attached to the second enclosure portion 190. The internal components of the high-throughput high-volume mechanoporation unit 100 may be the same as described above with reference to FIGS. 11A-11D.

FIG. 11F illustrates the high-throughput high-volume mechanoporation unit 100 of FIG. 11E with the first enclosure portion 110 removed. The intermediate gasket 145 is positioned within the second enclosure portion 190 with each of the intermediate gasket slots 146 having a separate processing unit 150. The second enclosure portion 190 may be sized smaller than the intermediate gasket 145 such that the second enclosure portion 190 compresses the intermediate gasket 145 and forms a seal between each processing unit 150 and the intermediate gasket 145 (as described above with reference to FIGS. 11A-11D).

FIG. 11G is a top schematic view of the second enclosure portion 190, illustrating the second-enclosure-portion cavity 193. The interior walls 195 forming the second-enclosure-portion cavity 193 may be tapered inward such that these interior walls 195 compress the intermediate gasket 145 and form a seal between each processing unit 150 as the intermediate gasket 145 and the processing units 150 are inserted into the second-enclosure-portion cavity 193. In some examples, the process of assembling the high-throughput high-volume mechanoporation unit 100 comprises (a) inserting the processing units 150 into the intermediate gasket slots 146 forming a subassembly, (b) inserting this subassembly into the second-enclosure-portion cavity 193, and (c) attaching the first enclosure portion 110 to the second enclosure portion 190.

### Media Examples

The cells may attempt to recover to their original volume as soon as no further compression forces are applied to them. In some cases, the methods of the present disclosure may comprise releasing the cells into a recovery space after compression by the compressive elements. In some cases, the cells may be immediately released into the recovery space. The recovery space may be inside the same cell processing apparatus. The recovery process may allow the cells to recover at least partially and to increase the volume by absorbing the surrounding media. The media may comprise one or more reagents, which may be introduced into the cells as a part of this recovery process. Examples of such reagents may comprise plasmids and magnetic nanoparticles (e.g., introduced into stem cells or other types of cells for various applications) and mRNA (e.g., introduced into primary peripheral blood mononuclear cells or other types of cells for various applications). Other reagents and cell types may be used.

As provided herein, one or more reagents may be directed into an interior of cells including large cells. The reagents may comprise large molecules. In some examples, the plurality of substances may have an average molecular weight greater than or equal to about 0.5 megadaltons (MDa), 0.6 MDa, 0.7 MDa, 0.8 MDa, 0.9 MDa, 1.0 MDa, 1.1 MDa, 1.2 MDa, 1.3 MDa, 1.4 MDa, 1.5 MDa, 1.6 MDa, 1.7 MDa, 1.8 MDa, 1.9 MDa, 2.0 MDa, 2.1 MDa, 2.2 MDa, 2.3 MDa, 2.4 MDa, 2.5 MDa, 2.6 MDa, 2.7 MDa, 2.8 MDa, 2.9 MDa, 3.0 MDa, 3.5 MDa, 4.0 MDa, 4.5 MDa, 5.0 MDa, or more. In some cases, each of the substances may have a molecular weight that is greater than or equal to about 0.5 megadaltons (MDa), 0.6 MDa, 0.7 MDa, 0.8 MDa, 0.9 MDa, 1.0 MDa, 1.1 MDa, 1.2 MDa, 1.3 MDa, 1.4 MDa, 1.5 MDa, 1.6 MDa, 1.7 MDa, 1.8 MDa, 1.9 MDa, 2.0 MDa, 2.1 MDa, 2.2 MDa, 2.3 MDa, 2.4 MDa, 2.5 MDa, 2.6 MDa, 2.7 MDa, 2.8 MDa, 2.9 MDa, 3.0 MDa, 3.5 MDa, 4.0 MDa, 4.5 MDa, 5.0 MDa, or more.

The reagents may or may not comprise a charged substance. For example, the reagents may comprise a drug, a nucleic acid molecule, an antigen, a polypeptide, an antibody, an antigen, a hapten, an enzyme, or combinations thereof. The nucleic acid molecule may comprise deoxyribonucleic acid (DNA), ribonucleic acid (RNA), peptide nucleic acid (PNA), or combinations thereof.

In some examples, the reagents comprise reagents greater than or equal to about 5 kilobases (kB), 6 kB, 7 KB, 8 kB, 9KB, 10 kB, 11 kB, 12 kB, 13 kB, 14 kB, 15 kB, 16 kB, 17 kB, 18 kB, 19 kB, 20 kB, 21 kB, 22 kB, 23 kb, 24 kB, 25 kB, 26 kB, 27 kB, 28 kB, 29 kB, 30 kB, or larger. One example reagent may be DNA plasmids with a size larger than 10 kB.

In some cases, large reagents, such as reagents at least about 10 kB in size may not be introduced by conventional microfluidic methods, such as squeezing cells in narrow pores to improve the membrane poration characteristics, followed by the slow diffusion of reagents through the temporary membrane pores. Since the diffusion is slower for larger reagents, such reagents may not be effectively delivered using these diffusion methods. Furthermore, the degree of cell membrane poration, proposed by conventional methods, may be limited to ensure cell viability and avoid cell damage or death. In some examples, conventional microfluidic devices for intracellular delivery may be prone to clogging because narrow channels may be used for achieving high membrane shear and opening membrane pores. Increasing flow rates, e.g., to reduce clogging, may result in cell damage.

### Conclusion

Although the foregoing concepts have been described in some detail for purposes of clarity of understanding, it will be apparent that certain changes and modifications may be practiced within the scope of the appended claims. It should be noted that there are many alternative ways of implementing processes, systems, and apparatuses. Accordingly, the present embodiments are to be considered illustrative and not restrictive.

1. A high-throughput high-volume mechanoporation unit for scalable and cost-effective cell therapy manufacturing, the high-throughput high-volume mechanoporation unit comprising:
a first enclosure portion comprising a media inlet;
a pre-processing media reservoir comprising a media cavity forming a delivery slot, wherein:
the media cavity is fluidically coupled with the media inlet, and
the pre-processing media reservoir is sealed against the first enclosure portion;
a processing stack comprising two end gaskets and a processing unit positioned between the two end gaskets, wherein:
each of the two end gaskets comprises a set of gasket slots, fluidically coupled to the delivery slot,
the processing unit comprises a set of inlet slots, a set of outlet slots, a set of processing channels extending between and fluidically coupling the set of inlet slots and the set of outlet slots, and a set of ridges extending into the set of processing channels and forming a set of gaps within the set of processing channels,
the set of inlet slots is aligned with the set of gasket slots in a first one of the two end gaskets, and
the second processing slot is aligned with one slot in the set of gasket slots in the second one of the two end gaskets;
a stack support interfaces the second one of the two end gaskets providing support to the processing stack when a pressure differential is applied across the processing stack; and
a second enclosure portion comprising a cavity fluidically coupled to the set of outlet slots and used for collecting the cell media after passing through the processing stack.

2. The high-throughput high-volume mechanoporation unit of claim 1, wherein each the set of gasket slots extends perpendicular to the delivery slot.

3. The high-throughput high-volume mechanoporation unit of claim 1, wherein:
the first one of the two end gaskets is positioned between and sealed against each of the processing unit and the pre-processing media reservoir, and
the second one of the two end gaskets is positioned between and sealed against each of the processing unit and the.

4. The high-throughput high-volume mechanoporation unit of claim 1, wherein:
the set of inlet slots does not overlap with the set of gasket slots in a second one of the two end gaskets, and
the second processing slot does not overlap with any slot in the set of gasket slots in the first one of the two end gaskets and is sealed by the first one of the two end gaskets.

5. The high-throughput high-volume mechanoporation unit of claim 1, wherein:
the stack support comprises a set of rib separated by a set of openings,
the set of rib provides support to the processing stack, and
the set of openings is aligned with the set of gasket slots in the second one of the two end gaskets.

6. The high-throughput high-volume mechanoporation unit of claim 1, wherein:
the second enclosure portion comprises a media outlet and a gas outlet, both fluidically coupled to the cavity, and
the gas outlet is located closer to the processing stack than the media outlet.

7. The high-throughput high-volume mechanoporation unit of claim 1, wherein the first enclosure portion comprises a first enclosure cavity such that the pre-processing media reservoir protrudes into the first enclosure cavity and seals to a bottom of the first enclosure cavity.

8. The high-throughput high-volume mechanoporation unit of claim 1, wherein:
the media cavity comprises a rectangular portion and a taper portion,
the rectangular portion faced and is fluidically coupled with the media inlet, and
the taper portion terminates with the delivery slot.

9. The high-throughput high-volume mechanoporation unit of claim 1, wherein:
the processing unit comprises a silicon base and a glass cover attached to the silicon base and forming the set of processing channels with the silicon base, and
the set of ridges is a part of the silicon base.

10. The high-throughput high-volume mechanoporation unit of claim 9, wherein the glass cover is attached to the silicon base using anodic bonding.

11. The high-throughput high-volume mechanoporation unit of claim 9, wherein:
the processing stack comprises a second processing unit comprising a second silicon base and a second glass cover attached to the second silicon base, and
the processing stack comprises an intermediate gasket providing a seal between the glass cover and the second silicon base.

12. The high-throughput high-volume mechanoporation unit of claim 11, wherein the intermediate gasket is formed by casting silicone rubber and compressed between the glass cover and the second silicon base.

13. The high-throughput high-volume mechanoporation unit of claim 11, wherein:
the glass cover comprises a planar surface directly interfacing the second silicon base, and
the glass cover further comprises a seal channel recessed from the planar surface and housing the intermediate gasket.

14. The high-throughput high-volume mechanoporation unit of claim 13, wherein:
the seal channel extends to an edge of the glass cover forming a set of seal channel openings, and
the intermediate gasket is formed in-situ by flowing a gasket material through the set of seal channel openings to fill the seal channel with the gasket material, while the planar surface of the glass cover directly interfaces the second silicon base, and curing the gasket material inside the seal channel.

15. The high-throughput high-volume mechanoporation unit of claim 14, wherein the gasket material is selected from the group consisting of a heat-curable material and a ultraviolet (UV) curable material.

16. The high-throughput high-volume mechanoporation unit of claim 9, wherein:
the processing unit comprises a processing-unit first surface formed by the glass cover and directly interfacing the first one of the two end gaskets,
the processing unit comprises a processing-unit second surface formed by the glass cover, and
each of the set of inlet slots and the set of outlet slots extends between the processing-unit first surface and the processing-unit second surface.

17. The high-throughput high-volume mechanoporation unit of claim 9, wherein:
one slot of the set of inlet slots extends between a first one of the set of outlet slots and a second one of the set of outlet slots,
a first subset of the set of processing channels extends between the one slot of the set of inlet slots and the first one of the set of outlet slots, and
a second subset of the set of processing channels extends between the one slot of the set of inlet slots and the second one of the set of outlet slots.

18. The high-throughput high-volume mechanoporation unit of claim 1, wherein each ridge of the set of ridges has a chevron shape with a leading chevron edge facing a corresponding one of the set of inlet slots.

19. The high-throughput high-volume mechanoporation unit of claim 1, wherein:
the stack support comprises a stack-facing surface that interfaces the second one of the two end gaskets, and
a set of openings taper away from the stack-facing surface.

20. A method of processing cells in a cell media using a high-throughput high-volume mechanoporation unit comprising a first enclosure portion, a pre-processing media reservoir, a processing stack, a stack support, and a second enclosure portion, the method comprising:
delivering the cell media to the pre-processing media reservoir;
increasing air pressure in the pre-processing media reservoir over the media to at least 5 psi (34 kPa) while monitoring an airflow rate through a gas outlet of the second enclosure portion;
reducing the air pressure in the pre-processing media reservoir to atmospheric level as soon the airflow rate through the gas outlet reaches at least a set limit; and
removing the media through a media outlet of the second enclosure portion.
